(19) United States
(12) Patent Application Publication
(10) Pub. No.: US 2011/0065782 A1
(43) Pub. Date: Mar. 17, 2011

(54) METHODS OF IDENTIFYING COMPOUNDS THAT INHIBIT THE ACTIVATION OF A BIOMOLECULE AND METHODS OF TREATMENT USING THE COMPOUNDS

(76) Inventors: Thérèse Malliavin, Paris (FR); Elodie Laine, Paris (FR); Arnaud Blondel, Paris (FR); Daniel Ladant, Cachan (FR); Johanna Karst, Thiais (FR); Sylvain Rault, Moult (FR); Aurélien Lesnard, Demouville (FR)

(21) Appl. No.: 12/559,638

(22) Filed: Sep. 15, 2009

(51) Int. Cl. A61K 31/38; C40B 30/00; C40B 30/02; C07D 333/36

(52) U.S. Cl. 514/447; 506/7; 506/8; 549/69

(57) ABSTRACT

The present invention relates to methods of identifying compounds that inhibit the activation between a biomolecule, pharmaceutical compositions comprising such compounds, and methods of treating and/or to reducing the risk of Bacillus anthracis and Bordetella pertussis infection by administering such pharmaceutical compositions.

### BACKGROUND OF THE INVENTION

(a) Field of the Invention

The present invention relates to methods of identifying compounds that inhibit the activation of a biomolecule, methods of using such compounds to treat and/or to reduce the risk of Bacillus anthracis and Bordetella pertussis infections, pharmaceutical compositions comprising such compounds, and kits comprising such compounds and pharmaceutical compositions.

(b) Description of the Related Art

Traditionally, new drugs have been identified by generating and screening thousands of candidate drug compounds. In structure-based drug design, however, the three-dimensional structure of the target molecule is used to guide drug discovery. In this approach, researchers start with a three-dimensional (3D) structure (e.g., a computer-generated 3D structure) of the biomolecule and then try to design drug candidates that will bind the biomolecule. This is akin to designing a key to fit a lock whose structure is known.

Structure-based drug design has been used to help identify numerous drugs (1-3). The success of this technique has been largely due to the growing number of computational resources (4-6), improved methods and models for conformational space sampling (7-10), and to the growth of databases containing biomolecular three-dimensional (3D) structures (11, 12).

Several structure-based drug design protocols have been developed (13-17), including virtual screening methods, which screen large numbers of known binding compounds (e.g., ligands, including small-molecule organic compounds) against the 3D structure of target biomolecules (e.g., proteins). Customarily, these virtual screening methods focus on identifying potential binding sites (i.e., pockets) within the biomolecules that the drug candidates can fit into. Most known screening methods, however, fail to take advantage of the dynamic nature of biomolecules. See, e.g., U.S. Pat. No. 7,386,398, incorporated by reference as if fully set forth herein in its entirety. For example, such methods often focus on targeting the active site of a biomolecule, but fail to target other pockets that may form when, for example, the biomolecule undergoes a conformational change as it binds a ligand. One disadvantage of targeting the active site of a biomolecule is that the site may be inaccessible due to steric considerations. In this case, even though the drug candidate could, in fact, fit into the active site, it may not be able to enter the active site to exert its therapeutic effect. Another disadvantage of targeting the active site of a biomolecule is that often times potential drug candidates are screened based on the volume size of the active site. But it has been reported that volume alone cannot predict whether a potential drug candidate will bind to a target site (18).

Accordingly, there is a need in the art for a virtual screening method that targets sites on biomolecules (e.g., sites other than the active site) that form as the biomolecule undergoes conformational changes. Such a method would target a larger number of potential binding sites and would consider a larger variety of potential drug candidates.

The present invention addresses this need. It provides methods of identifying allosteric sites on a biomolecule (i.e., sites other than the active site) that, when bound by a ligand, effect conformational changes (or prevents such changes from occurring) in the biomolecule thereby disrupting or inhibiting its ability to interact normally with other biomolecules. The invention also provides ligands useful in treating diseases caused when two biomolecules interact normally.

### SUMMARY OF THE PREFERRED EMBODIMENTS

In one aspect, the invention relates to a method for identifying one or more compounds that inhibit the activation of a biomolecule comprising:
(a) identifying an initial inactive conformation of the biomolecule, a final active conformation of the biomolecule and one or more intermediate conformations of the biomolecule between its initial and final conformation;
(b) identifying one or more pockets on the biomolecule, in said one or more intermediate conformations;
(c) screening said one or more pockets with a screening library of compounds; and
(d) selecting one or more compounds that inhibit the activation of the biomolecule.

In another aspect, the invention relates to a method for identifying one or more compounds that inhibit the activation between a biomolecule, said method comprising:
(a) determining a transition path describing the conformational change of the biomolecule during its activation, said transition path having an initial point and a final point, where the initial point is the inactive conformation of the biomolecule and the final point is the active conformation of the biomolecule;
(b) refining the transition path describing the conformational change of the biomolecule during its activation;
(c) identifying intermediate conformations of the biomolecule in the transition path;
(d) identifying one or more pockets on the biomolecule;
(e) screening said one or more pockets with a screening library of compounds; and
(f) selecting one or more compounds that inactivates the biomolecule.

In still another aspect, the invention relates to a method for reducing the risk of Bacillus anthracis infection comprising administering to a patient at risk of infection a therapeutically effective amount of a compound of the formula (I):

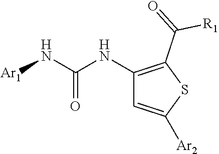

or a pharmaceutically acceptable salt thereof;

wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

In yet another aspect, the invention relates to a method for reducing the risk of Bordetella pertussis infection comprising administering to a patient at risk of infection a therapeutically effective amount of a compound of the formula (I):

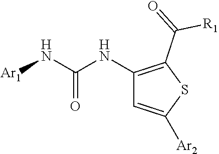

or a pharmaceutically acceptable salt thereof;

wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

In still another aspect, the invention relates to a complex between Edema Factor and a compound of the formula (I):

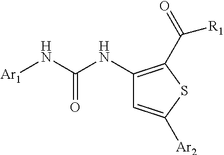

or a pharmaceutically acceptable salt thereof;

wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

In another aspect, the invention relates to a method of inhibiting Edema Factor (EF) comprising contacting EF with a compound of the formula (I):

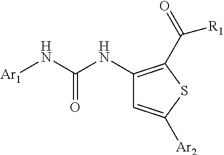

or a pharmaceutically acceptable salt thereof;

wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

In another aspect, the invention relates to a pharmaceutical composition comprising a compound of the formula (I):

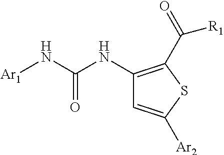

or a pharmaceutically acceptable salt thereof;

wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

In still another aspect, the invention relates to a kit comprising a pharmaceutical composition comprising a compound of the formula (I):

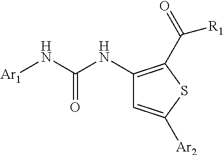

or a pharmaceutically acceptable salt thereof;

wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

### BRIEF DESCRIPTION OF THE DRAWINGS

FIG. 1 is a scheme illustrating the synthesis of thiophenyl ureido acids.

FIG. 2 is a plot showing the evolution of the transition path refinement for Edema Factor (EF). The plot shows the maximum energy (top left panel), the number of frames or intermediates states after reduction (bottom left panel), the path curvilinear length before reduction (top right panel) and the central processing unit (“CPU”) time used for a refinement cycle.

FIG. 3 is a plot showing the features of the final conformation transition path of EF: energy, energy gradient, distance (coordinate root mean square) to the starting point of the path, curvilinear length.

FIG. 4. is a plot showing projections onto the first normal modes of the principal component analysis (PCA) of the conformational transition path for the Edema Factor from CaM free conformation to that found in the calmodulin complex (EF-CaM). The projection of the successive path conformations are colored from light gray to black. The projection of the molecular dynamics (MD) simulations recorded on the EF-CaM complex in the presence of 0, 2 and 4 Ca²⁺ are also shown.

FIG. 5 depicts ribbon and cartoon representations of EF and shows the deformation of switches A, B, and C (SABC) along the transition path of EF. The SABC pocket was detected at the interface of switches A, B, and C (labeled): (a) top view of the surface of SABC in conf 1 (cartoon representation); (b-e) representation of SABC in conf 1 (b), 8 (c), 28 (d) and 47 (e) with the surface of the pocket displayed in transparent grey. Residues in the pocket are also shown.

FIG. 6 comprises graphs showing experimental inhibition percentages observed for the thiophenyl ureido acid compounds on EF. For each compound, the bars represent the control; the inhibition at 1, 10, and 100 μM; and the inhibition obtained on the pre-formed EF-CaM complex. The designations TUABr, TUACl, TUAF, TUAH, TUAdiCl, and TUAOCH3 correspond to the compounds disclosed in Table 1, below.

### DETAILED DESCRIPTION OF THE PREFERRED EMBODIMENTS

The present invention provides for methods of identifying ligands that inhibit the activation of a biomolecule. These methods involve the generation of a conformational path describing the transition between the biomolecule in its inactive form and the biomolecule in its active form. The methods further involve identification of relevant intermediate biomolecule conformers. Pockets may then be identified on the relevant biomolecule conformers. The pockets may be screened (e.g., in terms of their size, volume, and electronics) against known or prophetic ligands to identify ligands that bind to the pockets. The invention provides for compounds that inhibit the binding of particular biomolecules and their binding partners as well as methods of using such compounds to treat and/or to reduce the risk of infections by pathogens. In some embodiments, pathogens includes Gram-positive and Gram-negative bacteria. A non-limiting example of Gram-negative bacteria include Bordetella pertussis, Pseudomonas aeruginosa, Yersinia pestis, and Yersinia pseudotuberculosis. A non-limiting example of a Gram-positive bacterium includes Bacillus anthracis.

### DEFINITIONS

As used herein, a biomolecule is in its “active form” when its conformation allows a particular biological function. A biological function may be, for example, an enzymatic, signal transduction, molecular recognition, regulatory, cellular trafficking, molecular motors and the like.

A biomolecule in its “inactive form” when its conformation does not allow a particular biological function. In some embodiments, the biomolecule can be activated by its interaction with a binding partner. In other embodiments, a biomolecule can be inactivated by binding of an inhibitor to its active site (e.g., competitors ligands); binding a molecule to another site different of the active site that alters the active conformation (e.g., allosteric ligands); or modifying its conformation either by altering the physicochemical condition (e.g., pH, temperature, salt concentration).

As used herein, the term “ligand” refers to a molecule that associates or binds to a pocket on a biomolecule. The ligand can be a competitive ligand or an allosteric ligand. Ligands include biomolecules, as well as small-molecule organic compounds. In a preferred embodiment, the ligand includes small-molecule organic compounds such as the thiophenyl ureido acids described herein.

As used herein, the term “biomolecule” refers to any molecule exhibiting biological activity. Exemplary biomolecules include proteins, enzymes, and the like. A non-limiting example of biomolecules include adenylate cyclase toxins. Adenylate cyclase toxins include EF (Bacillus anthrasis), CyaA (Bordetella pertussis), ExoY (Pseudomonas aeruginosa), and the adenylate cyclase toxins of Yersinia pestis and Yersinia pseudotuberculosis. In some embodiments, the biomolecules have “binding partners.” As used herein, the term “binding partner” refers to a second biomolecule that can associate with a first biomolecule, thereby activating the first biomolecule. Thus, for example, the binding partner for EF and for CyaA is CaM.

As used herein, the terms “pocket,” “pockets,” and “binding pockets” refer to a region of a biomolecule or biomolecule complex, that, as a result of its configuration, favorably associates with or is occupied by an entity (e.g., a ligand) or region of the same biomolecule or biomolecule complex, or an entity or region of a different biomolecule, biomolecule complex, chemical compound or other compound. Typically, a binding pocket, or at least a portion thereof, comprises a cavity which is the site of interaction with an entity of the same or different molecule. As will be appreciated by those of skill in the art, the nature of the cavity within a binding pocket will vary from molecule to molecule.

As used herein, the term “complex” refers to the combination of two or more entities, at least one of which is a biomolecule. In particular, complexes in accordance with the present invention are formed between a biomolecule, including analogs thereof, which may include amino acid substitutions, truncations or insertions, and another compound. In some embodiments, the complex is between a biomolecule and it's binding partner. In other embodiments, the complex is between a biomolecule and a compound (e.g., ligand). The combination or “complexing” of a compound, chemical entity or binding partner with a biomolecule refers to the nature of the association/binding between the compound, chemical entity or binding partner and the biomolecule. The association between the components of the complex is the condition of proximity there between and may be covalent in nature. Alternatively, the association may be non-covalent in nature, wherein the juxtaposition is energetically favored by, e.g., hydrogen bonding, van der Waals forces or electrostatic interactions.

### Methods of Identifying Ligands

In one aspect, the invention relates to a method for identifying one or more ligands (e.g., small-molecule organic compounds) that inhibit the activation of a biomolecule. The method generally comprises:

(a) identifying an initial inactive conformation of the biomolecule, a final active conformation of the biomolecule and one or more intermediate conformations of the biomolecule between its initial and final conformation;

(b) identifying one or more pockets on the biomolecule, in said one or more intermediate conformations;

(c) screening said one or more pockets with a screening library of compounds; and

(d) selecting one or more compounds that inhibit the activation of the biomolecule

In some embodiments, the pockets identified at step (b) are other than the functional active site of the biomolecule.

In another aspect, the invention relates to a method for identifying one or more ligands (e.g., small-molecule organic compounds) that inhibit the activation of a biomolecule. The method generally comprises:

(a) determining a transition path describing the conformational change of the biomolecule during its activation, said transition path having an initial point and a final point, where the initial point is the inactive conformation of the biomolecule and the final point is the active conformation of the biomolecule;

(b) refining the transition path describing the conformational change of the biomolecule during its activation;

(c) identifying intermediate conformations of the biomolecule in the transition path;

(d) identifying one or more pockets on the biomolecule;

(e) screening said one or more pockets with a screening library of ligands; and

(f) selecting one or more ligand that inactivates the biomolecule

In another aspect, the invention relates to a method for identifying one or more allosteric ligands. In this particular embodiment, the step (d) comprises the identification of one or more pockets on the biomolecule, other than the functional active site of the biomolecule, in said intermediate conformations. In another aspect, the invention relates to a method for identifying one or more competitive ligands.

In yet another aspect, the invention relates to a method for identifying one or more compounds that inhibit the interaction between a biomolecule and its binding partner, comprising:

(a) identifying an initial conformation of the biomolecule without its binding partner, a final conformation of the biomolecule bound to its biomolecule and one or more intermediate conformations of the biomolecule between its initial and final conformation;

(b) identifying one or more pockets on the biomolecule, other than the functional active site of the biomolecule, in said one or more intermediate conformations;

(c) screening said one or more pockets with a screening library of compounds; and

(d) selecting one or more compounds that inhibit the interaction between the biomolecule and its binding partner.

In still another aspect, the invention relates to a method for identifying one or more compounds that inhibit the interaction between a biomolecule and its binding partner, said method comprising:

(a) determining a transition path describing the conformational change of the biomolecule during its interaction with its binding partner, said transition path having an initial point and a final point, where the initial point is the conformation of the biomolecule without its binding partner and the final point is the conformation of the biomolecule bound to its binding partner;

(b) refining the transition path describing the conformational change of the biomolecule during its interaction with its binding partner;

(c) identifying intermediate conformations of the biomolecule in the transition path;

(d) identifying one or more pockets on the biomolecule, other than the functional active site of the biomolecule, in said intermediate conformations;

(e) screening said one or more pockets with a screening library of compounds; and

(f) selecting one or more compounds that inhibit the interaction between the biomolecule and its binding partner.

1. Determining a Transition Path

In general, the first step in the methods for identifying one or more ligands that inhibit the activation of a biomolecule is the determination of a transition path describing the conformational change of the biomolecule during its activation. In a particular embodiment the activation is triggered by a binding partner. The transition path has an initial point and a final point. The initial point is generally the conformation of the biomolecule without its binding partner. The final point is generally the conformation of the biomolecule with its binding partner.

This initial point may be determined, for example, by modeling the biomolecule without ions or cofactors, with a molecular simulation program such as the Chemistry at Harvard Macromolecular Mechanics (CHARMM) package, version 29 (19) using a force field such as PARM19. Optionally, the solvent may be modeled implicitly by using levels of approximation. For example, in the simplest level of approximation, a distance-dependent dielectric and a force shift at a distance specific for the biomolecule in question may be combined in order to fit globally a sigmoidal shape (20) for the dielectric constant. A more sophisticated solvation model may optionally be used in the refinement phase of the conformational transition discussed in greater detail below. One non-limiting example of such a model may be based on the analytical continuum electrostatics (ACE2) potential (21).

2. Refining the Transition Path and Identifying Intermediate Conformations of the Protein

After selecting the initial point and the final point of the transition path, the next step involves refining the transition path. During the refinement process, intermediate conformations of the biomolecule are identified. In some embodiments, the conformation of the biomolecule at the initial point of the path (e.g., the biomolecule without its binding partner) and the conformation of the biomolecule at the final point of the path (e.g., the biomolecule bound to its binding partner) may be considered to be intermediate conformations of the biomolecule.

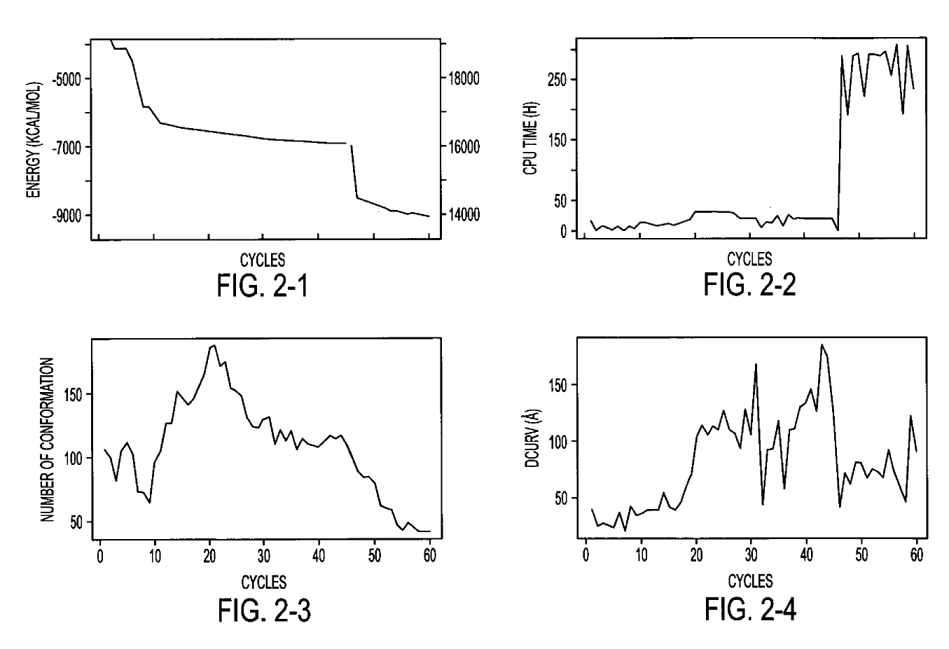

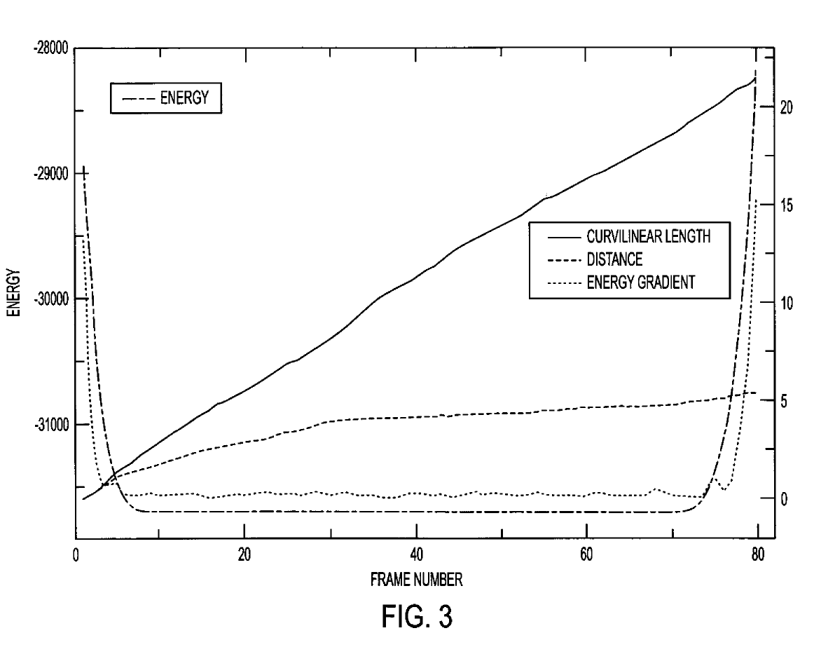

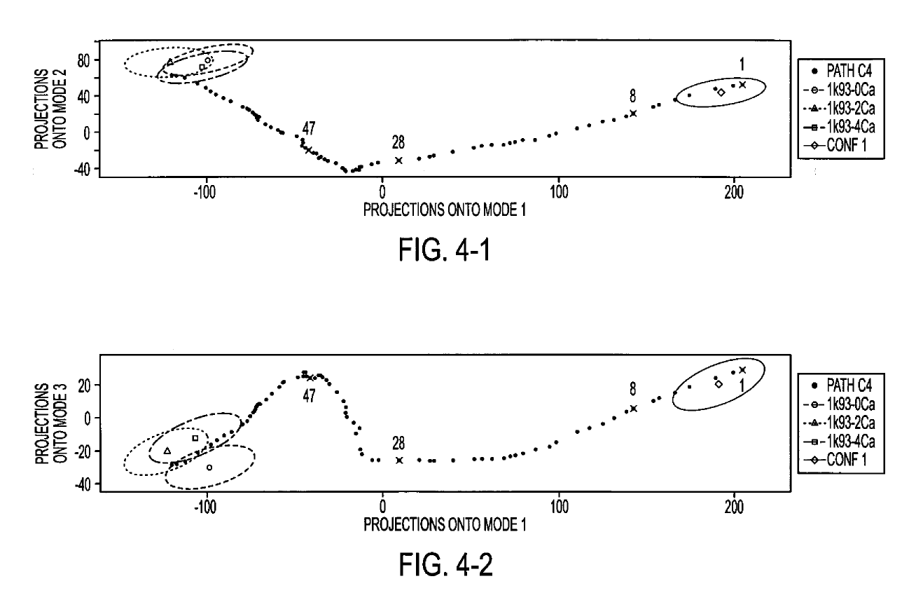

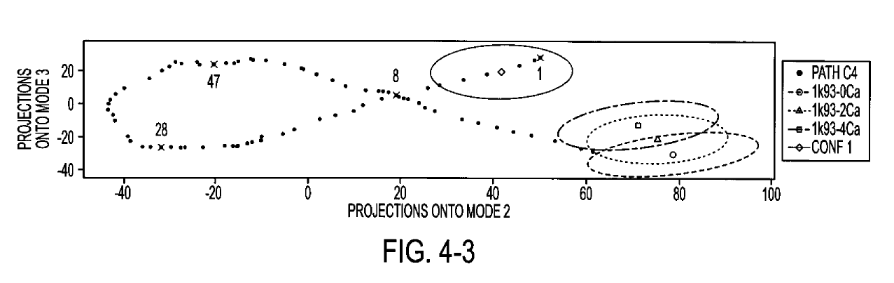

In a non-limiting example, the transition path may be determined by following the parameters: CPU time required for one refinement cycle; system energy; the number of frames or intermediates states in the path; and the curvilinear length of the path. As shown in FIG. 2, in the context of EF as a non-limiting example of the biomolecule, the CPU time (top right panel) and the energy (top left panel) display a transition around the cycle 45, when the solvation model is switched from a sigmoidal dielectric constant to the mode ACE2 (21). The energy (black curve) shows a systematic decrease along the path refinement within a region using the same solvation model. The number of frames or intermediate states in the path (bottom left panel) as well as the curvilinear length of the path display maximum values and a tendency to stabilize around smaller values. The path selected in the methods described herein may be the one with a region of stabilized energy, number of frames or intermediates states and, to a lesser extent, stabilized curvilinear length.

In some embodiments, the transition path may be further refined, in an iterative manner, by using two types of processing either separately or together: (i) a reduction of the number of conformations in the path; and (ii) a sampling of the conformations located between the path conformations. The purpose of this processing is to obtain a transition path, defined as a series of conformations linking the initial and final points of the path, in the frame of a force field, such as PARM19.

In other embodiments, the transition path may be further refined by:

a) reducing the number of intermediate conformations of the biomolecule in the transition path by retaining said intermediate conformations between which there is no energy barrier above a given threshold;

b) selecting a pair of intermediate conformations;

c) identifying one or more by-passes between said selected pair of intermediate conformations, wherein said by-passes comprise a new sequence of intermediate conformations;

d) selecting by-passes; and

e) assembling the selected by-passes.

In other embodiments, the transition path may be further refined by:

a) selecting a pair of intermediate conformations;

b) identifying one or more by-passes between said selected pair of intermediate conformations, wherein said by-passes comprise a new sequence of intermediate conformations;

c) reducing the number of intermediate conformations of the biomolecule in the transition path by retaining said intermediate conformations between which there is no energy barrier above a given threshold;

d) selecting by-passes; and

e) assembling the selected by-passes.

In some embodiments, step c) can occur after any one, two or after any three of steps a), b), d) or e); or step c) can occur after each of steps a), b), d), and e).

The reduction in the number of intermediate conformations is accomplished, in some embodiments, by iteratively truncating (for a given energy threshold) the sequence of intermediate conformations of the path between two predefined end conformations of a biomolecule. The predetermined end conformations could be known conformations of, e.g., a substrate, protein or the like, such as, for example, the active and inactive conformations of a the substrate, protein or the like. The truncation procedure starts by selecting the first predefined end conformation. The procedure is then run iteratively until the last predefined end conformation is reached. At each step of the truncation procedure, for a selected intermediate conformation, the farther intermediate structure in the sequence following that structure for which the energy threshold is still satisfied is identified. The energy threshold is satisfied if the energy of any conformation built by interpolation between a first selected intermediate conformation and the candidate farther intermediate conformation is below an energy threshold. By way of examples, an interpolated conformation between two given conformations can be obtained by linear interpolation of the Cartesian coordinates of those two given conformations. That identified intermediate structure is then considered to follow directly the selected intermediate structure in the sequence defining the path and the intermediate structures that were between are discarded. If no conformation can be identified at this step, the consecutive conformation can be used instead, to continue, by way of example. The identified conformation is then considered as the selected conformation in the next step of the iterative process.

As used herein, the term “by-pass” refers to a sub-path composed of a sequence of intermediate conformations connecting any selected pair of conformations. The selected pair of conformations forming the initial and terminal conformations of a given by-pass may be known conformations, such as, for example, the active and inactive conformations of a biomolecule, but they may also be any two intermediate conformations found within the sequence of intermediate structure of the path. The number of possible pairs of conformations, Nb, is Nb=Np*(Np+1)/2, for a path composed of a number, Np, of intermediate conformations. For example, Nb is 5050 when Np=100 and approximately half a million for Np=1000.

In some embodiments, a sub-path comprises a new sequence of intermediate conformations between a selected pair that can be different from that found previously between that selected pair, and in different number. Any method that generates new conformations can be used to generate a new sequence of intermediate conformations. Such methods include, without limitation, using molecular dynamics, Brownian dynamics, Monte-Carlo methods, other path calculation methods, and geometrical algorithms. In some embodiments, such methods can be used with or without additional constraint functions in addition to the score function.

Useful by-passes may be selected by evaluating the quality of their respective paths. Various criteria can be used to evaluate the quality of a by-pass. By-passes with better quality are either composed of fewer structures, shorter in curvilinear length, lower in energy, or satisfying a lower energy threshold or any combination of these properties. The curvilinear length, Cl, between any selected pair of intermediate conformations is the sum of the distances between all the pairs of consecutive intermediate conformations that can be found in the sequence of the series of conformations between the selected intermediate conformations. By-passes may be selected by choosing the by-pass where the number of enclosed conformations between a selected pair of conformations is lower than the number of enclosed conformations in a different by-pass between the same pair of conformations. By-passes may also be selected by selecting the by-pass between a selected pair of conformations satisfying a lower energy threshold than that of a different by-pass between the same pair of conformations. Still other methods of selecting by-passes may be employed, alone or in combination with one another.

In a path, the maximum energy, located between two selected intermediate conformations can be overcome with by-passes. By-passes can connect the selected intermediate conformations. Alternatively, the search for by-passes between a given pair of conformations can be extended beyond the selected pair of conformations to form a wider by-pass. For example, there could be four intermediate structures along a path, having the order A-B-C-D. Searching for by-passes between each intermediate structure may result in conformation X, which is between A and B; conformation Y, which is between B and C; and conformation Z, which is between C and D, such that the order is now A-X-B-Y-C-Z-D. If another by-pass was sought between B and C that would be similar but perhaps wider, a new by-pass could be created starting from X and ending at Z. Other wider by-passes could be sought between any pair of intermediate conformations (e.g., from B to Z, or even X to C). Useful wider by-passes are either composed of fewer structures, shorter in curvilinear length, lower in energy, or any combination of these properties. Useful wider by-passes can be generated by using, for example, molecular dynamics, Brownian dynamics, Monte-Carlo methods, other path calculation methods, and geometrical algorithms. In some embodiments, such methods can be used with or without additional constraint functions in addition to the score function.

The wider by-passes can be evaluated successively. All possible wider by-passes that can be defined can be characterized by the number of intermediate conformations that are enclosed between their selected ends (i.e., between the selected pair of intermediate conformations). In order to conserve and/or minimize the computational power required, all wider by-passes may be analyzed in a specific order, starting with the by-pass with the fewest number of intermediate structures and continuing through each successive by-pass and ending with the by-pass with the most intermediate structures. However, the search can be stopped when certain criteria are reached. For example, the energy, and more specifically the maximum energy along the by-pass can be one of those criteria. In some embodiments, by-passes with lower maximum energy are preferred. The curvilinear length and number of conformations composing the by-pass may be indicative of the variability between bypasses. Lower values in curvilinear length and number of conformations correspond to more preferred and/or higher quality paths. In some embodiments, the process of evaluating by-passes can be analyzed independently using a series of two or more computations running simultaneously and/or in parallel. Additional criteria that can be used to search for wider by-passes include, for example, the number of enclosed intermediate conformations (e.g., the minimum number, Nmin), the distance between the selected structures (e.g., the maximum distance, Dmax), and the ratio of distance to the curvilinear length RMS/Cl (e.g., the maximum ratio of RMS/Cl, Rmax). These additional criteria can be adjusted so that the total number of by-passes and/or the total number of intermediate conformations comprising the total set of bypasses corresponds to a reasonable amount of computational effort, based on existing computational hardware and/or computing power.

With a given set of intermediate conformations, constructing all possible by-passes and by-passes combinations represents a combinatorial problem of evaluating each individual conformation paired with every other conformation. In this manner, by-passes may be constructed between any two conformations, subject to any possible limitations on the number of intermediate conformations found on the by-pass, the maximum energy of the by-pass, or any other desired limit. For example, for the series of intermediate conformations A-B-C-D, possible by-passes could be A→B, A→C, B→D, and C→D where, for example, the annotation A-B represents the subseries of intermediate conformations between conformation A and B in the path [A . . . B], the annotation A→B represents the new series of intermediate conformations between A and B in the by-pass. An individual by-pass may be considered compatible with another by-pass if the selected pair of conformations serving as the terminal conformation of one by-pass is not inside the sequence by-passed in a different by-pass. For example, for the series of intermediate conformations A-B-C-D, the by-pass A→B would be compatible with the by-pass B→C or C→D, but A→C would not be compatible with B→D. Alternatively, two by-passes considered to be incompatible can sometimes be made compatible by assembling A→C-B→D and reduction, if in the sequences of A→C and B→D there exist two conformations X and Y respectively, such that X-Y satisfies the energy threshold, leading to A→X-Y→D in the reconstruction. Any combination of compatible by-passes can be assembled to reconstitute a new path, simply by replacing the corresponding intermediate conformations of the previous path by that of the compatible by-passes.

In some embodiments, an exhaustive incremental combinatorial construction of all the combinations of compatible by-passes can be implemented. Compatible by-passes can be combined to form new by-passes, which may be subjected to further refinement to yield new paths.

The number of such series of compatible by-passes, Ns, is rapidly increasing with the number of compatible by-passes (up to exponential growth). It may then be useful to iteratively select, by way of example, only same types of combinations in the number of by-passes used in the reconstruction to limit the number of by-pass combinations in the search to yield better combinations. The best combinations of by-passes can be selected based directly on the maximum energy, the number of intermediate structures and curvilinear length of the assembled path or that obtained after application of a procedure on the latter. However, to fully evaluate the benefit of a combination of by-passes which are close to each other, it is important to evaluate it as a whole by applying the procedure that is to be applied for the selection after assembly. In some embodiments, a combination of by-passes that is close to each other is typically by-passes separated by less than five intermediate conformations.

As used herein, the term “path” or “transition path” refers to a series of conformations where each pair of consecutive conformation satisfies a given energy threshold. In the case where a simple level of approximation is used, the path refinement drives the energy reduction, starting, for example from a value of from about −3,000 kcal/mol going down to about −7,000 kcal/mol, for example, −3,088 kcal/mol and going down to −6,914 kcal/mol. When the analytical continuum electrostatics (ACE2) potential is used, the energy reduction between each conformation drives the path refinement, starting, for example, from a value of from about −25,000 kcal/mol down to −32,000 kcal/mol, e.g., from about −30,650 kcal/mol (which corresponds to the lowest value calculated with the simple model) down to about −31,700, representing a decrease of hundreds to thousands kcal/mol or between 0.1 kcal/mol per atom to 10 kcal/mol per atom.

The processing step (i) (reduction of the number of conformations) may be preferably performed by truncating a given path in a iterative way. For each sub-path going from the conformation nₖ to the final point of the transition, one searches the largest index k′ such that the path going from nₖ to n_{k′} is in agreement with the energy threshold. The conformation n_{k′} then becomes the protein conformation following nₖ in the reduced path.

The processing step (ii) (sampling of the conformations located between two path conformers) may preferably be performed using an algorithm capable of finding reaction paths or accurate transition states, such as the Conjugate Peak Refinement (“CPR”) (22) or molecular dynamics, between pairs of conformations according to the following criteria: (a) the number of conformers in the path between the two selected conformations is larger than a given threshold Nmin, (b) the root mean square distance (RMSD) between the two conformations is lower than a given threshold Dmax, and (c) the ratio RMSD/D_{curv} is smaller than the given threshold Rmax, where D_{curv} is the curvilinear distance between the conformations defined as:

$${D}_{curv} = {\sum }_{k = 1}^{N - 1} RMSD ( {X}_{k} , {X}_{k + 1} )$$

where N is the number of the path conformers, Xₖ represents the coordinates of the kth conformer, and RMSD(Xₖ,Xₖ₊₁) is the coordinate RMSD between two successive conformers in the path.

In some embodiments, the criterion (b) may be used in order to limit the difficulty of finding by-passes satisfying the energy threshold, whereas the criteria (a) and (c) may be used to limit possible “detours” made by the transition path in the conformation space. These criteria may be preferably applied manually by the user according to the results obtained in the previous refinement cycle. Thus, for example, Nmin can be varied from 25 down to 5; Dmax can be varied between 1.5 up to 7 Å; and Rmax can be varied between 0.17 up to 0.7.

In some embodiments, each refinement cycle may comprise one path reduction and one step of conformer generation, by-pass and assembly (i.e., intermediate conformations of the biomolecule in the transition path), and may last, in some cases, up to hundreds of CPU hours. In some embodiments, after an appropriate number of refinement cycles (e.g., 2 for a simple transition up to about 100, preferably about 80 and most preferably 50 depending on the topological complexity of the transition), the electrostatic energy modeling the solvent may be switched from a simple approximation scheme, such as the sigmoidal-shaped dielectric constant model, to a more sophisticated solvation model, such as ACE2, in order to prevent the formation of voids inside the protein.

3. Identifying One or More Pockets

After refining the transition path and identifying intermediate conformations of the biomolecule in the transition path, the next step involves the identification of one or more pockets eventually other than the functional active site of the biomolecule in the intermediate conformations to which a ligand may bind. When the one or more pockets are pockets other than the functional active site of the biomolecule, such pockets can be considered allosteric sites.

In some embodiments, pockets may be identified at the surface of analyzed proteins using an algorithm designed to identify potential ligand binding locations on the surface of biomolecules, such as the PocketFinder module (23) of the ICM software package (24). In brief, the algorithm employed may make use of a carbon probe to create a van der Waals grid potential map. Potential ligand envelopes may be detected by contouring the map and may then be filtered by their volume, possibly using a lower limit of 100 Å³. The contouring level may be determined as:

contouring_level=mean(map)−threshold.rmsd(map)

where mean(map) and rmsd(map) are the mean and the standard deviation values of the potential map, calculated on all map values, and where the default threshold of 4.6 Å is sometimes used. Other algorithms designed to identify potential ligand binding locations on the surface of biomolecules include LIGSITE (B. Huang and M. Schroeder, BMC Structural Biology 6:19 (2006)) and PASS (B. Brady Jr. and P. Stouten, J. Computer Aideed Molecular Design 14:383401 (2000)).

In cases where the crystal structure of the biomolecule and the biomolecule bound to its binding partner is known (see, e.g., Published U.S. Patent Appl. No. 2008/0124746 for EF bound to CaM), a systematic search for pockets may be performed on the crystallographic structures, on the intermediate conformations of the transition path, and on representative conformations extracted from MD trajectories determined for the free biomolecule and for the biomolecule bound to its binding partner.

4. Targeting the Pocket with a Screening Library of Compounds

After the identification of the pockets, the next step involves screening the identified pockets for ligands using a screening library of compounds. In some embodiments, prior to targeting, it may be necessary to prepare the biomolecule for such targeting, such as selecting appropriated amino acids forming the pocket and which will contribute to interact with the prophetic ligand, adjusting the protonation of certain protonatable amino acid residues on the biomolecule in general and inside the identified pockets. For example, in some cases, it may be necessary to adjust histidine protonation for a receptor using computational methods for approximating macromolecular protonation sites. One such example may be based on geometric methods attempting to place protons based upon local hydrogen bonding environments or based upon optimization of hydrogen bonding networks, which may be accomplished using software such as WHATIF (25). Another such example may be based upon electrostatic methods attempting to place protons based upon electrostatic considerations or attempting to calculate or approximate pKa shifts of specific amino acids.

Once any necessary adjustments have been made, a pocket may be screened in silico against ligands for potential binding. In some embodiments, a library of compounds may be employed as potential ligands for such virtual screening. The virtual representations of the compounds must exist as three-dimensional (3D) representations prior to screening. Conversion of two-dimensional (2D) representations of compounds in the library into 3D representations, as well as adding hydrogens, converting to necessary formats, etc., may be accomplished by suitable programs, including a 3D structure generator such as CORINA (26, 27).

As used herein, the term “library of compounds” or “screening library of compounds” refers to a collection of compounds that is generated in-house by the user or a collection of compounds collected by a third-party. Third parties include, e.g., Centre d'Etudes et de Recherche sur le Médicament de Normandie, Chimiothèque Nationale (France) and corporate entities like Chembridge Corp., San Diego, Calif. and ChemDiv Inc., San Diego, Calif.

In some embodiments, the 3D structures of the compounds from the screening library of compounds may be screened for docking capability with a pocket using a docking algorithm that proceeds through incremental construction of the ligand inside the one or more pockets identified by the methods described herein. Such an algorithm may be driven by the search for maximized biomolecule-ligand interactions. An exemplary algorithm is the FlexX algorithm (13). The docking algorithm may generate a scoring function for evaluating the suitability of docking a given ligand with a given pocket. Examples of scoring functions include the FlexX standard scoring function, which is similar to the Bohm function (28). In some embodiments, the scoring function is used to select compounds in a first, inactive, conformation. In other embodiments, the scoring function is used to discard compounds in a second, intermediate, conformation.

In some embodiments, ligand-protein interactions may be extracted and classified by the docking algorithm into different types, including, without limitation: H-donor/H-acceptor, H-acceptor/H-donor, phenyl-center/phenyl-ring, phenyl-center/CH₃-phenyl, phenyl-center/amide, phenyl-ring/phenyl-center, CH₃-phenyl/phenyl-center, amide/phenyl-center, contact with the protein main chain, contact with the protein side-chain. Binary vectors, such as the 10 bit-long binary vectors produced by FlexX, obtained for the interactions of each of the residues defining the screened pocket, may then be concatenated to obtain a Structural Interaction Fingerprint

SIFt analysis (27, 29, 30) may consist of the characterization and clustering of the predicted docking poses of a putative ligand, based on the interactions made with the targeted biomolecule. Each docking pose may be represented by a Sift binary vector where each bit stands for the presence (1) or the absence (0) of a specific interaction between the ligand and one residue of the biomolecule binding site. In order to reduce the number of poses subject to SIFt analysis, in some embodiments, docking poses for each ligand may be clustered by the docking algorithm through an agglomerative hierarchical clustering (complete linkage method), which may include an arbitrary distance cutoff, such as 3 Å (31, 32).

In some embodiments, the similarity between two SIFts, A and B, may be evaluated from their Tanimoto coefficient T_{c} (33):

T_{c}(A,B)=|A∩B|/|A∪B|

where |A∩B| is the number of ON bits common in both A and B and |A∪B|s the number of ON bits present in either A or B. Interaction fingerprints may be clustered through an agglomerative hierarchical clustering approach (34). The construction, clustering and visualization of SIFts may be performed using appropriate software, such as R (35).

5. Selecting One or More Ligands

After screening the identified pockets with a screening library of compounds, the next step involves selecting one or more ligands that inhibit the activation of a protein. In some embodiments, ligands may be selected by using the scoring function values generated by the docking algorithm for each ligand and removing all ligands with scores highest than a threshold θ calculated as the mean best score μ over all ligands minus the standard deviation (θ>μ−σ; −12.6−4.7=−17.3). In a preferred embodiment, the top 1% of the remaining screened ligands may be selected for further screening and/or analysis.

In some embodiments, pockets identified on intermediate conformations of the biomolecule that lie along the transition path may be used as negative controls to filter potential false positive ligands. Ligands able to bind to the pockets on these intermediate conformations may be considered false positive. In this embodiment, potential ligands may be considered valid only if its mean scoring function value, minus the standard deviation over, e.g., multiple (e.g., 20) conformations, is better than the best scoring function values obtained for pockets identified on the intermediate conformations.

In some embodiments, a second filtering screen may be applied through the re-docking of selected ligands with a second docking algorithm, potentially one which makes use of a more accurate representation of the system based on an alternative molecular mechanics force field, such as the ICM software (24) and its accompanying force fields (14, 36). In some cases, the second score range may be found to be quite different from the first score for each considered ligand. In some embodiments, ligands with second scores deviating from the first by a given threshold amount may be discarded. For example, if the ICM score is positive or greater than, e.g., −10 kcal/mol, the ligand may be discarded.

Ligands selected using the methods described above may be further evaluated for their ability to inhibit the activation of a biomolecule by using standard in vitro methods known in the art. Some in vitro methods are described in the Example. Such an evaluation can sometimes be done, e.g., by measuring the interaction between a biomolecule and its binding partner.

Methods of Identifying Compounds that Inhibit the Interaction Between Edema Factor and Its Binding Partner Calmodulin

The Edema Factor (EF) from Bacillus anthracis is activated as an adenyl cyclase through its interaction with calmodulin (CaM) (37) and has recently been shown to impair the host immune system by inhibiting the expression of anthracidal phospholipase A2 by macrophages (38). During the interaction with CaM, EF undergoes a large conformational transition from a closed to an open conformation, the transition leading to the formation and activation of its adenylyl cyclase catalytic site, which transforms ATP into cAMP.

The EF-CaM complex is an example of an induced-fit interaction between proteins, as the two partners display a large conformational transition before the interaction takes place. An extensive study of the complex by molecular dynamics simulation (39, 40) has made it possible to obtain a better understanding of the dynamics and energetics of the interaction.

The invention provides for a method of identifying one or more compounds that inhibit (or disrupt) the interaction between EF and its binding partner CaM. The method targets a pocket selected from a conformational path describing the transition that EF undergoes in the presence of CaM. The path was produced using a original protocol based on an extensive use of Conjugate Peak Refinement (CPR) (22) and on an efficient way of sorting the conformations obtained by CPR. This method is detailed in the Example.

Although this section describes the specific example of EF as the biomolecule and CaM as its binding partner, the skilled artisan will recognize that the methods described herein for identifying one or more compounds that inhibit the activation of a biomolecule and/or the methods for identifying one or more compounds that inhibit the interaction between a biomolecule and its binding partner can be employed on other biomolecules and other binding partners.

Compounds that Inhibit EF

The experimental testing of 18 inhibitors identified using the methods described herein revealed six compounds having a common thiophenyl ureido acid scaffold. Prior to the present disclosure, compounds having a thiophenyl ureido acid scaffold were not known to inhibit EF enzymatic activity. The compounds identified by the methods provided herein are thiophenyl ureido acid compounds of the formula (I):

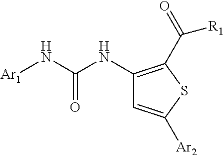

or a pharmaceutically acceptable salt thereof;

wherein:
Ar₁ is an optionally substituted aromatic ring;
Ar₂ is an optionally substituted aromatic ring, where Ar₁ and Ar₂ are the same or different; and
R¹ is hydroxy, alkoxy, arylalkyleneoxy, or heteroarylalkyleneoxy.

As used herein, the term “an optionally substituted aromatic ring” or “aryl” refers to a group that has at least one ring having a conjugated pi electron system and includes both carbocyclic aryl (e.g., phenyl) and heterocyclic aryl (or “heteroaryl”) groups (e.g., pyridine). The term includes monocyclic or fused-ring polycyclic (i.e., rings which share adjacent pairs of carbon atoms) groups. The aromatic ring is optionally substituted with one or more group(s) individually and independently selected from hydroxy, alkoxy, mercapto, alkylthio, arylthio, cyano, halo, nitro, and amino, including mono- and di-substituted amino groups.

As used herein, the term “alkoxy” refers to an alkyl-O— group, wherein “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, non-branched, or cyclic. The alkyl moiety includes alkylene moieties; that is, an alkyl group that is not terminal (e.g., a CH₃). Non-limiting examples of “alkylene” include, e.g., methylene and ethylene groups. Non-limiting examples of “arylalkylene” include, e.g., a benzyl group. A non-limiting example of an “arylalkyleneoxy” group includes a benzyloxy group. And a non-limiting example of an “heteroarylalkyleneoxy” group includes a pyridinylmethyloxy group.

Preferably, the alkyl group has 1 to 20 carbon atoms, e.g., a medium size alkyl having 1 to 10 carbon atoms and a lower alkyl having 1 to 4 carbon atoms. The alkyl group may be substituted or unsubstituted. When substituted, the substituent group(s) is(are) preferably one or more group(s) individually and independently selected from cycloalkyl, aryl, heteroaryl, heteroalicyclic, hydroxy, alkoxy, aryloxy, mercapto, alkylthio, arylthio, cyano, halo, nitro, and amino, including mono- and di-substituted amino groups. Non-limiting examples of alkyl groups are methyl, ethyl, propyl, i-propyl, n-butyl, t-butyl, sec-butyl, pentyl, and the like.

As used herein, the term “alkylthio” refers to an alkyl-S— group, wherein “alkyl” is as defined above.

As used herein, the term “arylthio” refers to an aryl-S— group, wherein “aryl” is as defined above.

As used herein, the term “cycloalkyl” refers to an unsaturated cyclic alkyl group having from 3 to 7 carbon atoms. Non-limiting examples of cycloalkyl groups include cyclopropyl, cyclobutyl, cyclopentyl, and cyclohexyl groups. The cyclic group optionally comprises one or more heteroatoms (e.g., nitrogen, oxygen, and sulfur). Non-limiting examples of cyclic rings having one or more heteroatoms include pyrrolidinyl, piperidinyl, and piperazinyl groups.

As used herein, the term “halo” or “halogen” refers to an atom selected from the group consisting of fluorine, chlorine, bromine, and iodine.

As used herein, the term “amino” refers to an —NR₂R₃ group, where R₂ and R₃ are, independently, hydrogen or alkyl.

As used herein, the term “pharmaceutically acceptable salt” refers to a formulation of a compound that does not abrogate the biological activity and properties of the compound. Pharmaceutical salts can be obtained by reacting a compound with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

Solvates and prodrugs of compounds of the formula (I) are also contemplated herein. The term “solvate” refers to a compound or a salt thereof, that further includes a stoichiometric or non-stoichiometric amount of solvent bound by non-covalent intermolecular forces. For example, where the solvent is water, the solvate is a hydrate.

The term “prodrug” refers to a derivative of a compound that can hydrolyze, oxidize, or otherwise react under biological conditions (in vitro or in vivo) to provide an active compound, particularly a compound of formula (I). Examples of prodrugs include, but are not limited to, derivatives and metabolites of a compound of formula (I), including biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers Gmfh).

As used herein, and unless otherwise indicated, the terms “biohydrolyzable amide,” “biohydrolyzable ester,” “biohydrolyzable carbamate,” “biohydrolyzable carbonate,” “biohydrolyzable ureido,” “biohydrolyzable phosphate” mean an amide, ester, carbamate, carbonate, ureido, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, alkoxyacyloxy esters, alkyl acylamino alkyl esters, and choline esters. Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

The compounds of the formula (I) can comprise chiral centers. Accordingly, enantiomers and diastereomers of compounds of the formula (I) are contemplated herein. Enantiomers are chemical compounds whose molecular structures have a non-superimposable mirror-image relationship to each other. A diastereomer is a stereoisomer of a compound having two or more chiral centers that is not a mirror image of another stereoisomer of the same compound.

In some embodiments compounds of the formula (I) are compounds where Ar₁ is phenyl. In other embodiments, Ar₂ is phenyl. In still other embodiments, Ar₂ is substituted with one or more halogen groups or one or more alkoxy groups. Preferred alkoxy groups are methoxy groups. Preferred halogen groups are bromine, chlorine and fluorine. In yet other embodiments, R₁ is hydroxy or alkoxy. In some preferred embodiments, R₁ is hydroxy.

Specific compounds falling under formula (I) that have been identified are compounds of the formula:

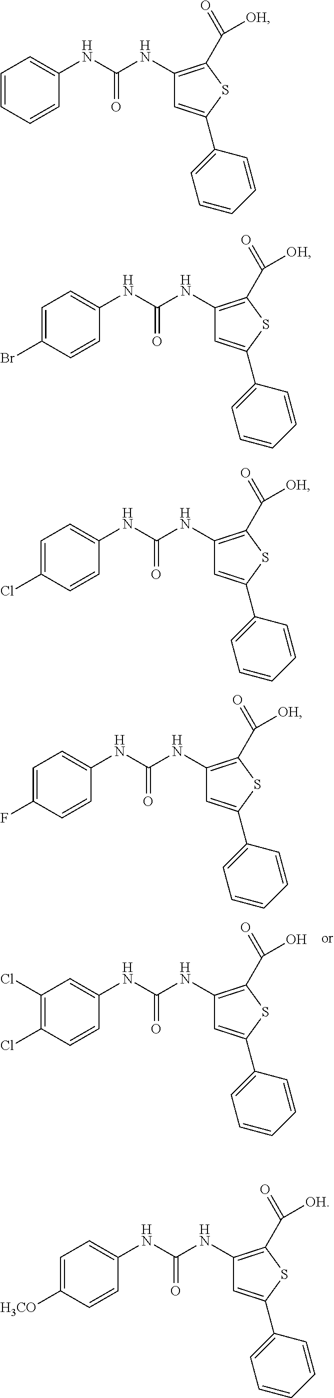

Of these compounds, the compound of the formula

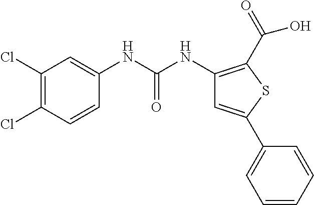

was found to inhibit EF activity at a concentration of less than 10 μM. Accordingly, compounds of the formula (I) are believed to be effective in methods of treating Bacillus anthracis infections.

The six specific compounds described above that fall under the formula (I) have also been assayed on CyaA, the adenyl cyclase of Bordetella pertussis. CyaA has been shown to have a mode of activation similar to EF, even though CyaA displays distinct structural features (41). Of the compounds described above, the compound of the formula

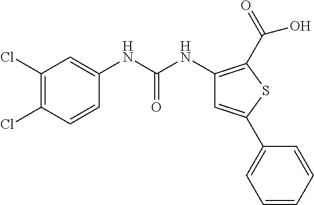

was found to inhibit CyaA activity, thus leading to the conclusion that compounds of the formula (I) will also be effective in methods of treating and/or reducing the risk of Bordetella pertussis infections.

In some embodiments, a complex between EF and a compound of the formula (I) is contemplated. In some embodiments, a complex between CyaA and a compound of the formula (I) is contemplated.

### Pharmaceutical Compositions, Administration, and Kits

A compound of the formula (I), or a pharmaceutically acceptable salt thereof, can be administered to a subject (e.g., human) in need thereof by itself or it can be administered in pharmaceutical compositions in which the compound is mixed with suitable carriers or excipient(s). Techniques for formulation and administration of drugs may be found in “Remington's Pharmacological Sciences,” Mack Publishing Co., Easton, Pa., latest edition.

As used herein, “administer” or “administration” refers to the delivery of a compound of formula (I), or a pharmaceutically acceptable salt thereof, or of a pharmaceutical composition containing a compound of formula (I), or a pharmaceutically acceptable salt thereof, to an organism for the purpose of treating and/or to reduce the risk of Bacillus anthracis or a Bordetella pertussis infection. As used herein, the term “treating” includes abrogating, substantially inhibiting, slowing or reversing the progression of a condition or substantially ameliorating clinical symptoms of a condition.

In some embodiments, the method described herein further comprise the step of selecting or obtaining a subject who has already been infected or is at risk of infection by Bacillus anthracis or a Bordetella pertussis The subject is selected from those individuals who have been infected by Bacillus anthracis or a Bordetella pertussis, yet are asymptomatic or individuals who have been infected by Bacillus anthracis or a Bordetella pertussis and exhibit one or more symptoms of anthrax or whooping cough. Subjects “at-risk” include those individuals who are asymptomatic but are more likely than the bulk of the population to develop the disease, because of personal or family history, behavior, exposure to disease causing agents or some other reason.

Suitable routes of administration may include, without limitation, oral, rectal, topical, transmucosal or intestinal administration or intramuscular, subcutaneous, intramedullary, intrathecal, direct intraventricular, intravenous, intravitreal, intraperitoneal, intranasal, or intraocular injections. The preferred routes of administration are oral and parenteral.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethylcellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also.

The capsules may be packaged into brown glass or plastic bottles to protect the active compound from, e.g., light.

For administration by inhalation, the compounds for use according to the present invention are conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, a compound of formula (I), or a pharmaceutically acceptable salt thereof, or a pharmaceutical composition containing a compound of formula (I), or a pharmaceutically acceptable salt thereof, may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (e.g., subcutaneously or intramuscularly) or by intramuscular injection. A compound of formula (I), or a pharmaceutically acceptable salt thereof, or a pharmaceutical composition containing a compound of formula (I), or a pharmaceutically acceptable salt thereof, may be formulated for this route of administration with suitable polymeric or hydrophobic materials (e.g., in an emulsion with a pharmacologically acceptable oil), with ion exchange resins, or as a sparingly soluble derivative such as, without limitation, a sparingly soluble salt.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions may also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Pharmaceutical compositions suitable for use in the present invention include compositions wherein the compound of formula (I), or a pharmaceutically acceptable salt thereof, or a pharmaceutical composition containing a compound of formula (I), or a pharmaceutically acceptable salt thereof, are contained in an amount sufficient to achieve the intended purpose (i.e., a therapeutically effective amount), e.g., treating and/or reducing the risk of Bacillus anthracis or a Bordetella pertussis infection.

More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of, for example, EF activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition.

Dosage amount and interval may be adjusted individually to provide plasma levels of the active species which are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, e.g., between 30-90% and between 50-90% of the time.

In cases of local administration or selective uptake, the effective local concentration of the drug may not be related to plasma concentration and other procedures known in the art may be employed to determine the correct dosage amount and interval.

The amount of a composition administered will, of course, be dependent on the subject being treated, the severity of the affliction, the manner of administration, the judgment of the prescribing physician, etc.

The compositions may, if desired, be presented in a pack or dispenser device, such as an FDA approved kit, which may contain one or more unit dosage forms containing the active ingredient (e.g., a compound of formula (I), or a pharmaceutically acceptable salt thereof, or a pharmaceutical composition containing a compound of formula (I), or a pharmaceutically acceptable salt thereof). The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition.

In some embodiments, a compound of formula (I), or a pharmaceutically acceptable salt thereof, or a pharmaceutical composition containing a compound of formula (I), or a pharmaceutically acceptable salt thereof, can be combined with other agents that are conventionally used for the treating and/or to reducing the riskof Bacillus anthracis or a Bordetella pertussis infection. Such agents include, but are not limited to, ciproflaxin and erythromycin, respectively.

The following examples are not intended to limit the invention in any way.

### EXAMPLES

### Example 1

### Identification of Compounds that Inhibit the Interaction of Edema Factor and Calmodulin

### Materials and Methods

### Chemical Ligands Tested

The compounds used in these examples were obtained from the chemical library of the CERMN (Centre d'Etudes et de Recherche sur le Médicament de Normandie).

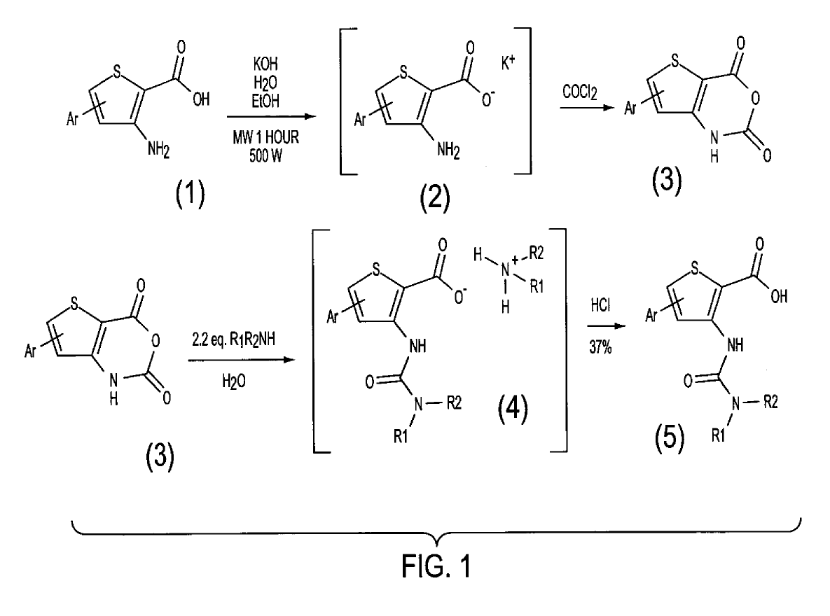

The library of 1140 thiophenyl ureido acids was synthesized as shown generally in FIG. 1 by the reaction of a 60 primary or secondary amines with a number of 19 thieno[3,2-(1]- or thieno[2,3-d][1,3]oxazine-2,4-diones. The compounds were obtained by a simple solution-phase combinatorial strategy on a 200-400-mg scale. The yields were over 70% yields and the purities over 80%.

The synthesis of anhydrides (FIG. 1, compound 3) started from the corresponding aminoesters (FIG. 1, compound 1) prepared following a Kirsch method for 4-substituted aminoesters (42, 43) or an Arnold-Vilsmeier-Haack (44, 45) method for the 5-substituted aminoesters. The alkaline hydrolysis of these aminoesters under microwave heating conditions led to the non-isolated aminocarboxylate intermediates (FIG. 1, compound 2), and phosgene addition permitted isolation of the anhydrides (FIG. 1, compound 3) in high yields (73-82%). The anhydrides reacted in a water suspension with 2.2 equivalent of a primary or secondary amine, leading to water-soluble ammonium ureido thiophene (FIG. 1, compound 4) carboxylates. Acidification of the reaction mixture permitted recovery of the thiophenyl ureido acids (FIG. 1, compound 5) as a precipitated solid after filtration. Washing with water discarded excess of amine as a water soluble hydrochloride.

### Biochemical Assay of Compounds

Compounds were dissolved in DMSO to a 10 mM stock concentration and kept frozen at −20° C. until use. The proteins, EF, CyaA and calmodulin (CaM) were purified as previously described (46-18). Escherichia coli inorganic pyrophosphatase (PPiase) was purchased from Sigma (ref 15907) and the Pi-ALS kit from Innova Biosciences (Innova Biosciences Ltd, Babraham Hall, Cambridge UK).

### Adenylate Cyclase Enzymatic Assay

To characterize the inhibitory potential of the different compounds toward the bacterial adenylate cyclase (AC) toxins, a colorimetric assay for adenylyl cyclase activity in microplate format was designed. AC converts ATP into cAMP and inorganic pyrophosphate (PPi) that can be further cleaved by pyrophosphatase (PPiase, e.g., from E. coli, ref Sigma 15907) into 2 phosphate (Pi) molecules. The Pi molecules can be detected colorimetrically by the change in absorbance (read at 595 nm) of the dye malachite green in the presence of phosphomolybdate complexes.

The assays were carried out as follows. EF was diluted into the AC assay buffer (20 mM Hepes-Na, pH 7.5, 15 mM MgCl₂, 0.2 mM CaCl₂, 0.01% TWEEN 20 (v/v) 20 (v/v) supplemented with 4 units/ml of PPiase (1 unit of PPiase hydrolyzes 1 μmol of PPi per minute) to a final concentration of 0.2-0.5 nM and 25 mL were distributed in the wells of a 96 well microtiter plates. Compounds (10 μL from a 5× concentrated solutions in assay buffer) were added to the indicated final concentrations (i.e. 100, 10 or 1 μM) and incubated for 20 min at 22° C. Then calmodulin (CaM, 5 μL from a 20 μM stock solution in assay buffer) was added to a final concentration of 2 μM and the mixtures were further incubated for 10 min at 30° C. The enzymatic reaction was initiated by addition of ATP (10 μL, 2 mM final concentration) and the microplate was incubated at 30° C. under agitation. Ten μL samples from each well were then taken out at different incubation times (from 3 to 15 min) and transferred into a second microtiter plate containing, in each well, 100 μL of a Pi-ALS mixture made of 10 μL of H₂O, 80 μL of Pi ColorLock ALS reagent and 10 μL Accelerator solution (both provided in the Pi-ALS kit from Innova Biosciences). The enzymatic reaction was immediately stopped by the acidic conditions of this Pi-ALS mixture. After 5 min of incubation at room temperature, 10 μL of stabilizer solution (from the Pi-ALS kit) were added to prevent further nonenzymatic breakdown of the phosphorylated substrate under acidic conditions (according to the kit instructions). After a further 30-60 min of incubation at room temperature, the Optical Densities at 595 nm (OD₅₉₅) were recorded with a microplate reader (Tecan, Lyon, France).

A standard curve was performed in parallel by adding known concentrations of Pi into Pi-ALS mixture and used to convert OD₅₉₅ values into moles of PPi produced. The enzymatic activity was calculated from the initial velocity of PPi synthesis (in the above described conditions, the accumulation of PPi was linear with time). In the absence of compounds, the specific activity of EF was found to be between 1 and 2 mmol PPi per min and mg of protein (corresponding to 100% activity). When CaM was omitted in the assay mixture, the PPi synthesis activity was below the detection level (i.e. <1 μmol PPi per min and mg of protein, corresponding to 0% activity). In certain experiments (see section “Results”), CaM was mixed with EF prior to the addition of the compounds.

Bordetella pertussis CyaA was assayed similarly at a final concentration of 0.1-0.2 nM. CyaA specific activity was 2.5 mmol PPi per min and mg of protein in the presence of CaM and below the detection level (i.e., <1 μmol PPi per min and mg of protein) in the absence of CaM.

### CaM-Binding Properties Probed by Steady-State Anisotropy Fluorescence

A 22 amino-acid long peptide, P₂₃₃₋₂₅₄, corresponding to the main CaM-binding sequence of CyaA (49) and encompassing residues 233 to 254 (in one letter code, LDRERIDLL-WKIARAGARSAVG with an acetylated Nₜₑᵣ), was synthesized by Genosphere Biotechnologies (Paris, France). Calcium-dependent binding of the P₂₃₃₋₂₅₄ peptide to CaM was monitored by measuring the steady-state anisotropy fluorescence of its unique Tryptophan residue (CaM has no tryptophan). The assay was carried out with an FP-6200 spectrofluorimeter (Jasco, Japan) in a Peltier-thermostated cell holder, using a 1 cm path length quartz cell (101.QS from Hellma). The excitation wavelength was fixed at 295 nm and emission spectra were recorded at 25° C. from 300 to 400 nm. Bandwidths of 5 and 10 nm were used for excitation and emission beams, respectively. Tryptophan anisotropy of peptide P₂₃₃₋₂₅₄ (2 μM) was measured with or without CaM (2 μM) and/or TUAdiCl (10 μM) in buffer A (20 mM Hepes, 150 mM NaCl, pH 7.4) supplemented with 0.2 mM CaCl₂ or 2 mM EGTA. Steady-state fluorescence anisotropy experiments were performed with vertically (V) and horizontally (H) polarized beam light using FDP-223 polarizers at both excitation (x) and emission (m) apertures. Anisotropy values (r), measured at a wavelength of 345 nm, were calculated as follows:

$$r = \frac{{V}_{x} {V}_{m} - \frac{{H}_{x} {V}_{m}}{{H}_{x} {H}_{m}} {V}_{x} {H}_{m}}{{V}_{x} {V}_{m} + 2 \frac{{H}_{x} {V}_{m}}{{H}_{x} {H}_{m}} {V}_{x} {H}_{m}}$$

### The Crystallographic Structures

Four crystallographic structures of EF were used corresponding to the structure of the EF-CaM complex (37) (PDB ids: 1K93, 1K90, 1XFX) and of the free EF (PDB id: 1K8T). The crystallographic CyaA structure analyzed corresponds to the CyaA conformation in interaction with the C-terminal part of CaM (35) (PDB id: 1 YRT).

### Molecular Dynamics Simulations

The molecular dynamics (MD) simulation were performed using the SANDER module from AMBER 8 and the PMEMD module of AMBER 10 (50). The MD simulations performed on the complex EF-CaM loaded with 0, 2 and 4 ions Ca²⁺ were described elsewhere (39). An additional 15 ns MD simulation was performed on the free EF in a closed conformation, using the same setup, except that the temperature was regulated using a Langevin thermostat (52) with a collision frequency of 2 ps⁻¹.

Refinement of the Conformational Transition Path

The EF chain was modeled alone, without ions or cofactors, with CHARMM version 29 (19) and with the force field PARM19. The solvent was modeled implicitly using two levels of approximation. In the simplest one, a distance-dependent dielectric and a shift at a distance of 8 Å are combined, in order to fit globally a sigmoidal shape (20) for the dielectric constant. The van der Waals interaction is modeled by applying a switch between cutoff distances of 7 and 8 Å. A more sophisticated solvation model was used in the second refinement phase of the conformational transition (see below). This model is based on the analytical continuum electrostatics (ACE2) potential (21). The dielectric constant was 1 inside the protein, and 80 outside. A force shift at the distance 10-12 Å is used to complement the ACE2 model. See, e.g., reference (21).

The initial and final points of the conformational transition (i.e., the initial point of the path and the final point of the path) were chosen as the conformation C of the structure 1K93 for the complex EF-CaM loaded with 2 ions Ca²⁺, and the conformation of the structure 1K8T for the free EF. As the loop 289-299 (switch B) present in 1K93 was not visible in the crystallographic structure deposited in 1K8T, a model loop was built into 1K8T by translating into this structure the conformation of the corresponding loop observed in 1K93 and application of simulated annealing. The internal coordinates of the two extreme conformations were then compared in order to detect sidechain flips. The flips were inspected manually in order to exclude unnecessary flips and crankshaft motions. The sidechain orientations were manually made similar in both protein states, in the cases where this operation was not breaking meaningful hydrogen bonds. This flips adjustments apply to the following amino acids: F, Y, D, E (strict symmetry) and Q, N, H (pseudo symmetry).

The initial step of the transition path determination was two Steered Molecular Dynamics (SMD) simulations; one starting from the initial point of the path and one starting from the final point of the path. For each point, the coordinate RMSD to the other path extremity was used to calculate the driving force of the simulation, with a force constant of 0.5 kcal/mol/A. The simulations were run at a temperature of 300K using a Langevin thermostat (51) with a friction coefficient of 100 ps⁻¹. The timestep was 1 fs, 100,000 steps of SMD were performed, and the conformations were saved every 200 steps to produce 500 conformations for each simulation.

The sets of conformations sampled during the two SMD simulations represented the initial guess of the conformational transition path. This path was further refined in an iterative way by alternating two types of processing: (i) a reduction of the number of conformations in the path and (ii) a sampling of the conformations located between the path conformations. The purpose of this processing was to obtain a transition path, defined as a series of conformations linking the terminal predefined conformations, in the frame of the CHARMM force field. To be a transition path, the series of conformations should verify that every path conformation or any conformation obtained by linear interpolation between path conformation has an energy smaller than a given threshold. The threshold reduction drives the path refinement, starting from a value of −3,065 kcal/mol down to −6,915 kcal/mol when a simple level of approximation is used and going to −30,650 kcal/mol down to −31,700 kcal/mol when an analytical continuum electrostatics (ACE2) potential is used

The processing step (i), reduction of the number of conformations, was performed by truncating a given path in a iterative way. For each sub-path going from the conformation nₖ to the final point of the transition (the open EF conformation), one searches the largest index k′ such that the path going from nₖ to n_{k′} is in agreement with the energy threshold. The conformation n_{k′} becomes the protein conformation following nₖ in the reduced path.

The processing step (ii), sampling of the conformations located between two path conformers, is performed using the algorithm of the Conjugate Peak Refinement (CPR) (34) between pairs of conformations respecting several criteria: (a) the number of conformers in the path between the two selected conformations is larger than a given threshold Nmin, (b) the RMSD between the two conformations is larger than a given threshold Dmax, and (c) the ratio RMSD/D_{curv} is smaller than the given threshold Rmax, where D_{curv} is the curvilinear distance between the conformations defined as:

$${D}_{curv} = {\sum }_{k = 1}^{N - 1} RMSD ( {X}_{k} , {X}_{k + 1} )$$

where N is the number of the path conformers, Xₖ represents the coordinates of the kth conformer, and RMSD(Xₖ,Xₖ₊₁) is the coordinate RMSD between two successive conformers in the path.

The criterion (b) is used in order to limit the difficulty of finding reaction paths or accurate transition states (e.g., performing a CPR search), whereas the criteria (a) and (c) intend to limit possible detours made by the transition path in the conformation space. These criteria were applied manually by the user, according to the results obtained in the previous refinement cycle. During the CPR algorithm, the atoms were prevented to move more than 0.5 Å. For example, Nmin can be varied from 25 down to 5; Dmax can be varied between 1.5 up to 7 Å; and Rmax can be varied between 0.17 up to 0.7.

Wherein EF transition some secondary structure elements switch from beta sheets to alpha helix. Therefore a few dozens of refinement cycles were applied to sort major topological changes prior to the application of an elaborated solvation model necessary to better model pockets. Each refinement cycle comprised one path reduction and one step of conformers generation, and last about 300 hours CPU on Intel Xeon® X5355 64 bits at 2.60 GHz. After 42 refinement cycles, the electrostatics energy was switched from the sigmoidal dieletric to the model ACE2, in order to prevent incorrect modeling of pockets inside the protein. At the end of the refinement, the path used in the following to perform virtual screening, was composed of 80 conformations, having a total curvilinear length of 80.7 Å, and the energy reduction threshold was −29,000 kcal/mol.

### In Silico Screening and Analysis

Pockets were identified at the surface of analyzed proteins using the PocketFinder module (23) of the ICM software package (24), which aims at identifying “druggable” ligand binding sites at the surface of proteins. In brief, the algorithm makes use of a carbon probe to create a van der Waals grid potential map. Potential ligand envelopes are detected by contouring the map and are then altered by their volume using a lower limit of 100 Å³. The contouring level was determined as:

contouring level=mean(map)−threshold.rmsd(map)

where mean(map) and rmsd(map) are the mean and the standard deviation values of the potential map, calculated on all map values, and where the default threshold of 4.6 Å was used.

A systematic search for pockets was performed on the crystallographic structures of EF 1K93 and remodeled 1K8T (37, 52), on the 80 intermediate conformations of the transition path, and on representative conformations extracted from MD trajectories recorded on the EF-CaM complex (39) and on free EF.

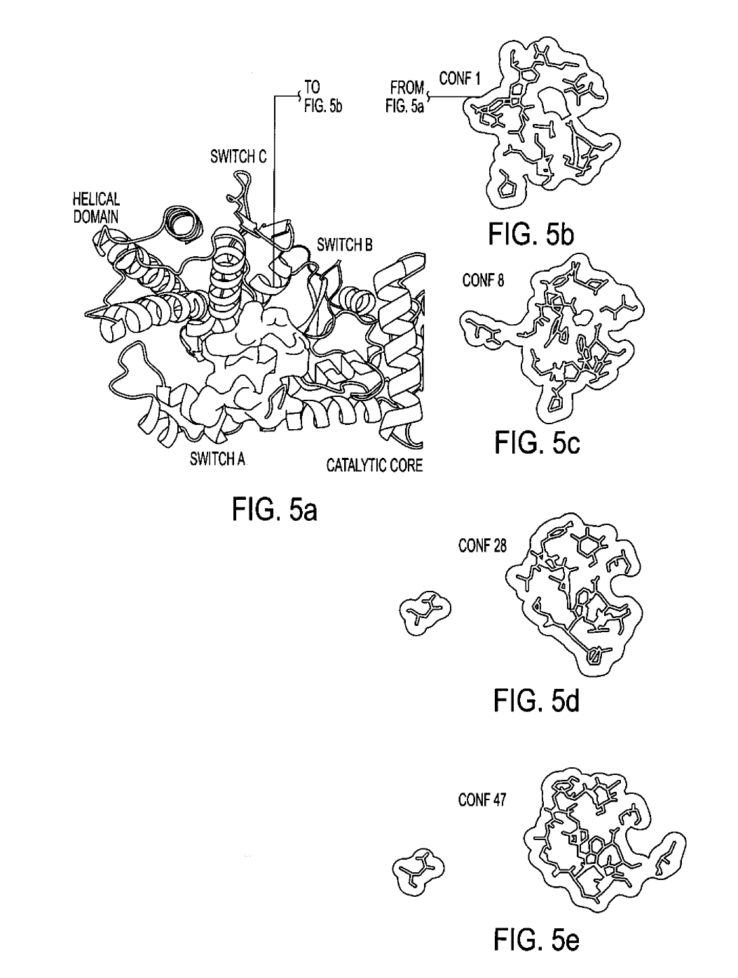

Ten potential ligand binding sites were identified, including the catalytic site. At the surface of the closed conformation of EF, a cavity of 450 Å³ was found enclosed by switches A, Gln C, hereinafter the SABC pocket. All 3 switches play a critical role in EF activation, either through direct contacts with CaM allowing for its insertion, or through stabilization of the catalytic site (37, 52). Hence they undergo large rearrangements during the transition, which modifies the shape of the SABC pocket as can be seen on FIG. 5. While the cavity of the pocket is well formed in the early steps of the transition path (conf 1, FIG. 5a, 5b), it disappears progressively upon CaM insertion (FIG. 5c, 5d, 5e) because of: (i) the increasing gap between the helical domain (residues 657-768) and the catalytic region (residues 291-656), (ii) the backbone rearrangements of switches A and C, (iii) the side-chains reorientation of residues in contact with CaM in the complex.

### Virtual Screening

Virtual screening of the CERMN library containing 28,329 molecules at the time (36) was performed with FlexX (53) onto the SABC pocket defined as residues: Ala 496, Pro 499, Ile 538, Glu 539, Pro 542, Ser 544, Ser 550, Trp 552, Gln 553, Thr 579, Gln 581, Leu 625, Tyr 626, Tyr 627, Asn 629, Asn 709. The initial conformation conf 1 of the transition pathway was used as the target conformation. A first pruning of the database was carried out by removing all molecules with scores worse than a threshold θ calculated as the mean best score μ over all compounds minus the standard deviation (θ=μ−σ=−12.6−4.7=−17.3. The 1% best ligands of the 3649 remaining compounds were selected as potential hits.

Table 1, below, shows the structures of six of the compounds identified.

TABLE 1

| Compound designator | R | % Inhibition |
|---|---|---|
| TUABr | 4-Br | 101 ± 1 |
| TUACl | 4-Cl | 98 ± 3 |
| TUAF | 4-F | 102 ± 2 |
| TUAH | H | 96 ± 7 |
| TUAdiCl | 3,4-di-Cl | 98 ± 6 |
| TUAOCH3 | 4-OCH₃ | 96 ± 7 |

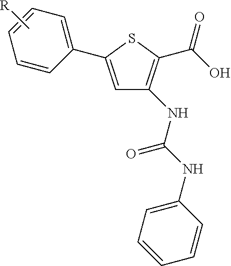

Three intermediate conformations conf 8, 28 and 47 picked up along the transition path were then used as negative controls to filter out false positives. Compounds able to bind to the distorted forms of the SABC pocket, observed at the surface of these conformations, as well as to the well-shaped pocket found in the initial conformation conf 1 were considered false positive. The strategy adopted was to search for ligands able to maintain the initial shape of the pocket. Potential hits were considered valid only if their mean score minus their standard deviation over 20 conformations was better than the best scores for conf 8, 28 and 47. Using this method, three compounds were eliminated from the set (Table 1). A second filter was applied through re-docking of the selected ligands with the ICM software (52), which makes use of a more accurate representation of the system based on a molecular mechanics force field (14, 36). ICM score range was found quite different from FlexX score for each considered ligand (Table 1). Ligands with ICM scores positive or greater than −10 kcal/mol were discarded. Overall 28 ligands were finally selected and 18 could be tested in vitro.

### In Vitro Characterization of the Putative EF Inhibitors

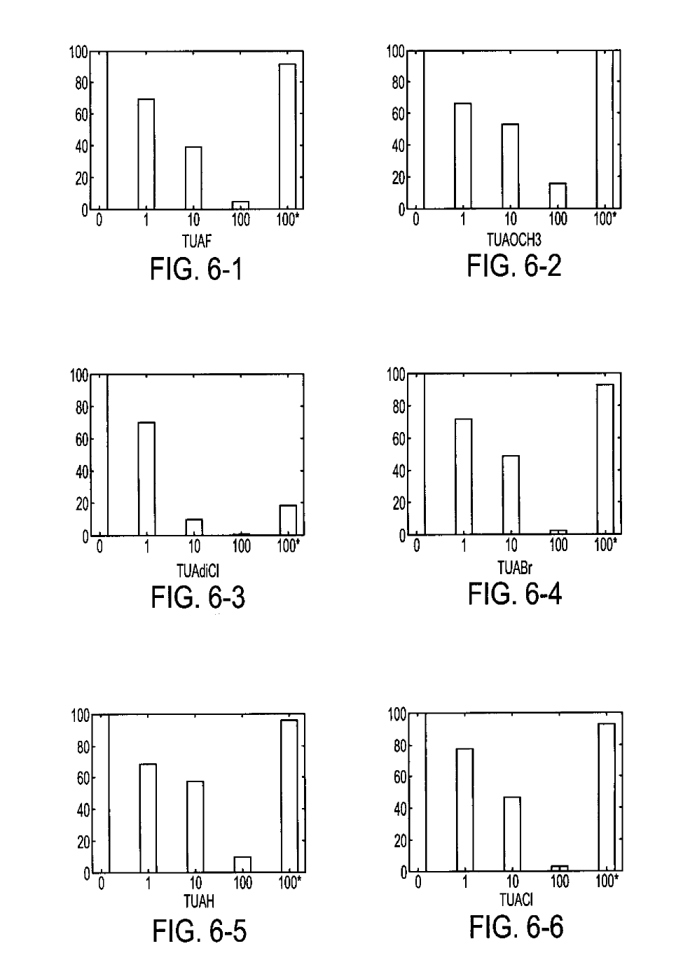

The inhibitory potential of the 18 available compounds toward the EF enzymatic activity was characterized experimentally. In the initial screening assays, EF (at 0.5 nM concentration) was preincubated with 100 μM of the compounds, before addition of CaM (2 μM final concentration). After an additional 10 min incubation, to allow the potential formation of the active EF-CaM complex, the enzymatic activity was determined by using a colorimetric assay that measured the inorganic pyrophosphate (PPi), that is released upon ATP conversion into cAMP. As shown in FIG. 6 and Table 1, a series of six highly related compounds, having a common thiophenyl ureido acid moiety, displayed a strong inhibitory capacity in these conditions. When tested at lower concentrations, however, only compound TUAdiCl still exhibited a significant inhibitory potency at 10 μM (>80% inhibition) with an overall IC₅₀ (i.e., concentration of inhibitors required for 50% inhibition) of 2-3 μM. Hence, TUAdiCl is a potent inhibitor of EF while the highly related thiophenyl ureido acid compounds were not as effective as TUAdiCl.

The effect of the six compounds was then examined in a different setup where EF was preincubated with CaM to form the EF-CaM complex prior to the addition of the compounds. As the EF-CaM complex is very stable (ref. and data not shown), it was expected that molecules that could prevent the association of EF with CaM would not be inhibitory on the preformed complex. This was indeed the case for five out of six compounds studied (FIG. 6). However, and quite unexpectedly, the high-affinity inhibitor TUAdiCl was found to be still able to significantly inhibit the enzymatic activity of the preformed EF-CaM complex.

These data suggest that among the compounds that were selected in silico to target the CaM-binding pocket, a series of molecules (Table 1) was indeed able to inhibit CaM-binding and activation of EF. The most potent member of this related family of compounds, TUAdiCl, exhibited an important inhibitory potency on CaM-bound EF suggesting that it might have an additional or a differential mode of inhibition relative to the other compounds studied.

To further explore the selectivity of these six selected compounds, they were also tested for their inhibitory potency against a related enzyme, the adenylate cyclase toxin (CyaA) from Bordetella pertussis, the causative agent of whooping cough. This toxin is also activated by CaM and the structure of its catalytic site bound to CaM revealed a high degree of structural homology with the EF-CaM complex, while their respective CaM contact surfaces are quite distinct (41).

The six compounds studied were found to be indeed potent inhibitor of the CyaA adenylate cyclase activity when tested at 100 μM (Table 1). However, at a 10 fold lower concentration, no significant inhibition were observed.

CaM is known to be the target for a numbers of small hydrophobic molecules that can block its interaction with target enzymes. Accordingly, experiments were conducted to determine whether the six thiophenyl ureido acid compounds studied might act through direct binding to CaM rather than to the EF enzyme. In these experiments, the effect of the most potent TUAdiCl compound was studied on the ability of CaM to bind, in a calcium-dependent manner, a 23 aminoacid peptide, P₂₃₃₋₂₅₄, corresponding to the main CaM-binding sequence of CyaA (46). Binding was monitored by following the changes in fluorescence anisotropy (r) of the unique tryptophan of the P₂₃₃₋₂₅₄ peptide (CaM has no tryptophan). The free peptide (2 μM) exhibited an anisotropy value of 0.03 (in the presence of calcium) that increased to 0.094 upon addition of an equimolar concentration of CaM, as a result of peptide binding to CaM. These values were not affected when the assays were carried out in the presence of 10 μM TUAdiCl (r=0.027 and 0.095 without and with CaM, respectively). This indicates that TUAdiCl, at a concentration that almost completely inhibited EF activity, had no effect on the ability of CaM to associate with a specific CaM-binding peptide from CyaA. It would appear from these data that the inhibitory potency of TUAdiCl toward EF activity resulted specifically from a direct action on the EF enzyme and not from an inhibition of the CaM activator.

Additional experiments performed on the compounds identified by the methods described herein suggest that those compounds do not bind to the catalytic pocket of EF. These experiments also suggest that these compounds all bind to EF via the same binding mode. In addition, these experiments suggest that these compounds bind to CyA through a mode similar to how they bind to EF. Finally, additional molecular dynamics experiments reveal an interplay between the shapes of the SABC pocket and of the catalytic pocket of EF.

All publications (e.g., Non-Patent Literature), patent application pulications, and patent applications mentioned in this specification are indicative of the level of skill of those skilled in the art to which this invention pertains. All such publications (e.g., Non-Patent Literature), patent application publications, and patent applications are herein incorporated by reference to the same extent as if each individual publication, patent, patent application publication, or patent application was specifically and individually indicated to be incorporated by reference.

### REFERENCES

[1] D. B. Kitchen, H. Decornez, J. R. Furr, and J. Bajorath. Docking and scoring in virtual screening for drug discovery: methods and applications. Nat Rev Drug Discov, 3:935-349, 2004.
[2] Z Zhou, A K Felts, R A Friesner, and R M Levy. Comparative performance of several flexible docking programs and scoring functions: enrichment studies for a diverse set of pharmaceutically relevant targets. J Chem Inf Model, 47:1599-1608, 2007.
[3] I Pauli, L F Timmers, R A Caceres, M B Soares, and W F de Azevedo. In silico and in vitro: identifying new drugs. Curr Drug Targets, 9:1054-1061, 2008.
[4] I. T. Arkin, H. Xu, M. O. Jensen, E. Arbely, E. R. Bennett, K. J. Bowers, E. Chow, R. O. Dror, M. P. Eastwood, R. Flitman-Tene, B. A Gregersen, J. L. Klepeis, I. Kolossváry, Y. Shan, and D. E. Shaw. Mechanism of Na+/H+ Antiporting. Science, 317:799-803, 2007.
[5] C Hadley. Biologists think bigger. EMBO Rep, 5:236-238, 2004.
[6] M R Shirts and V S Pande. Screen savers of the world, unite! Science, 290:1903-1904, 2000.
[7] E I Shakhnovich. Protein design: a perspective from simple tractable models. Fold Des, 3:R45-58, 1998.
[8] K Tai. Conformational sampling for the impatient. Biophys Chem, 107:213-220, 2004.
[9] A K Mazur and R A Abagyan. New methodology for computer-aided modelling of biomolecular structure and dynamics. 1. non-cyclic structures. J Biomol Struct Dyn., 6:815-032, 1989.
[10] R A Abagyan and A K Mazur. New methodology for computer-aided modelling of biomolecular structure and dynamics. 2. local deformations and cycles. J Biomol Struct Dyn., 6:833-845, 1989.
[11] H. M. Berman. The Protein Data Bank: a historical perspective. Acta Cryst A, 64:88-95, 2008.
[12] J M Shin and D H Cho. PDB-Ligand: a ligand database based on PDB for the automated and customized classification of ligand-binding structures. Nuc Acids Res, 33:D238-D241, 2005.
[13] M. Rarey, B. Kramer, T. Lengauer, and G. Klebe. A fast flexible docking method using an incremental construction algorithm. J. Mol. Biol., 261:470-489, 1996.
[14] R. A. Abagyan, M. M. Totrov, and D. A Kuznetsov. Icm: A new method for protein modeling and design: Applications to docking and structure prediction from the distorted native conformation. J. Comp. Chem., 15:488-506, 1994.
[15] R. A. Friesner, J. L. Banks, R. B. Murphy, T. A. Halgren, J. J. Klicic, D. T. Mainz, M. P. Repasky, E. H. Knoll, M. Shelley, J. K. Perry, D. E. Shaw, P. Francis, and P. S. Shenkin. Glide: a new Approach for rapid, accurate docking and scoring. 1. Method and assessment of docking accuracy. J Med Chem, 47:1739-1749, 2004.
[16] M. L. Verdonk, J. C. Cole, M. J. Hartshorn, C. W. Murray, and R. D. Taylor. Improved Protein-Ligand Docking Using GOLD. Proteins, 52:609-623, 2003.
[17] G. M. Morris, D. S. Goodsell, R. S. Halliday, R. Huey, W. E. Hart, R. K. Belew, and A. J. Olson. Automated docking using a Lamarckian genetic algorithm and an empirical binding free energy function. J Comput Chem, 19:1639-1662, 1998.
[18] A Buschiazzo, M Goytia, F Schaeffer, W Degrave, W Shepard, C Gre´goire, N Chamond, A Cosson, A Berneman, N Coatnoan, P M Alzari, and P Minoprio. Crystal structure, catalytic mechanism, and mitogenic properties of trypanosoma cruzi pro-line racemase. Proc Natl Acad Sci USA, 103:1705-1710, 2006.
[19] B. R. Brooks, R. E. Bruccoleri, B. D. Olafson, D. J. States, S. Swaminathan, and M. Karplus. Charmm—a program for macromolecular energy, minimization, and dynamics calculations. J Comp Chem, 4:187-217, 1983.
[20] R. Layery, H. Sklenar, K. Zakrzewska, and B. Pullman. The flexibility of the nucleic acids: (ii). the calculation of internal energy and applications to mononucleotide repeat dna. J Biomol Struct Dyn, 3:989-1014, 1986.
[21] M Schaefer, C Bartels, and M Karplus. Solution conformations and thermodynamics of structured peptides: molecular dynamics simulation with an implicit solvation model. J Mol Biol, 284:835-848, 1998.
[22] S Fischer and M Karplus. Conjugate peak refinement: an algorithm for finding reaction paths and accurate transition states in systems with many degrees of freedom. Chemical Physics Letters, 194:252-261, 1992.
[23] J. An, M. Totrov, and R. Abagyan. Comprehensive identification of druggable protein ligand binding sites. Genome Inform, 15:31-41, 2004.
[24] R Abagyan. ICM Manual v.3.1. Molsoft LLC, La Jolla, Calif., 2005.
[25] G. Vriend. WHAT IF: a molecular modeling and drug design program. J Mol Graph, 8:52-56, March 1990.
[26] J. Sadowski, J. Gasteiger, and G. Klebe. Comparison of automatic three-dimensional model builders using 639 x-ray structures. Journal of Chemical Information and Computer Sciences, 1994.
[27] S C Brewerton. The use of protein-ligand interaction fingerprints in docking. Curr Opin Drug Discov Devel, 11:356-364, 2008.
[28] H J Böhm. The development of a simple empirical scoring function to estimate the binding constant for a protein-ligand complex of known three-dimensional structure. Journal of Computer-Aided Molecular Design, 8:243-256, 1994.
[29] Z Deng, C Chuaqui, and J Singh. Structural interaction fingerprint (SIFt): a novel method for analyzing three-dimensional protein-ligand binding interactions. J Med Chem, 47:337-344, 204.
[30] G Marcou and D Rognan. Optimizing fragment and scaffold docking by use of molecular interaction fingerprints. J Chem Inf Model, 47:195-207, 2007.
[31] M. Rarey, S. Wefing, and T. Lengauer. Placement of medium-sized molecular fragments into active sites of proteins. J. Comput.-Aided Mol. Design, 10:41-54, 1996.
[32] R O. Duda and PE. Hart. Pattern Classification and Scene Analysis. John Wiley & Sons, Inc., New York, USA, 1973.
[33] P. Willett. Chemical similarity searching. J. Chem. Inf. Comput. Sci., pages 993-996, 1998.
[34] J A. Hartigan. Clustering Algorithms. Inc. New York, N.Y., USA, 1975.
[35] R Ihaka and R Gentleman. R: A Language for Data Analysis and Graphics. J. Comp. Graph. Statist., 5:299-314, 1996.
[36] M. Totrov and R. Abagyan. Flexible protein-ligand docking by global energy optimization in internal coordinates. Proteins, Suppl 1:215-220, 1997.
[37] C. L. Drum, S. Z. Yan, J. Bard, Y. Q. Shen, D. Lu, S. Soelaiman, Z. Grabarek, A. Bohm, and W. J. Tang. Structural basis for the activation of anthrax adenylyl cyclase exotoxin by calmodulin. Nature, 415:396 102, January 2002.
[38] B Raymond, D Leduc, L Ravaux, R L Goffic, T Candela, M Raymondjean, P L Goossens, and L. Touqui. Edema Toxin Impairs Anthracidal Phospholipase A2 Expression by Alveolar Macrophages. PLoS Pathog., 3:e187, 2007.
[39] E. Laine, J. D. Yoneda, A. Blondel, and T. E. Malliavin. The conformational plasticity of calmodulin upon calcium complexation gives a model of its interaction with the oedema factor of Bacillus anthracis. Proteins, 71:1813-1829, June 2008.
[40] E. Laine, A. Blondel, and T. E. Malliavin. Dynamics and energetics: a consensus analysis of the impact of calcium on EF-CaM protein complex. Biophys J, page in press, 2009.
[41] Q. Guo, Y. Shen, Y. S. Lee, C. S. Gibbs, M. Mrksich, and W. J. Tang. Structural basis for the interaction of Bordetella pertussis adenylyl cyclase toxin with calmodulin. EMBO J., 24:3190-3201, September 2005.
[42] G. Kirsch, D. Cagniant and P. Cagniant, Synthesis of 3-aminothiophenes from aryl- and hetarylacetonitriles, J. Heterocycl. Chem., 1982, 19 (2), 443-445.
[43] Rault, J C. Lancelot, B. Letois, M. Robba and Y. Labat, Preparation of 3-mercapto-2-thiophenecarboxylic acid derivatives as intermediates for herbicides, French Patent, No 9203732, Chem. Abstr., 1995, 123, 111841
[44] J. Liebscher, B. Neumann and H. Hartmann, Eine einfache Synthese von-Chlorzimtsäurenitrilen nach einer modifizierten Vilsmeier-Haack-Arnold-Reaktion, J. Prakt. Chem., 1983, 325(6), 915-918.
[45]H. Hartmann and J. Liebscher, A Simple Method for the Synthesis of 5-Aryl-3-amino-2-alkoxycarbonylthiophenes, Synthesis, 1984, 275-276.
[46] Y. Shen, Y. S. Lee, S. Soelaiman, P. Bergson, D. Lu, A. Chen, K. Beckingham, Z. Grabarek, M. Mrksich, and W. J. Tang. Physiological calcium concentrations regulate calmodulin binding and catalysis of adenylyl cyclase exotoxins. EMBO J., 21:6721-6732, December 2002.
[47] S Vougier, J Mary, N Dautin, J Vinh, B Friguet, and D Ladant. Essential role of me-thionine residues in calmodulin binding to Bordetella pertussis adenylate cyclase, as probed by selective oxidation and repair by the peptide methionine sulfoxide reductases. J Biol Chem, 279:30210-30218, 2004.
[48] G Karimova, C Fayolle, S Gmira, A Ullmann, C Leclerc, and D Ladant. Charge-dependent translocation of Bordetella pertussis adenylate cyclase toxin into eukaryotic cells: implication for the in vivo delivery of CD8+ T-cell epitopes into antigen-presenting cells. Proc Natl Acad Sci USA, 95:12532-12537, 1998.
[49] D. Ladant, S. Michelson, R. Sarfati, A. M. Gilles, R. Predeleanu, and O. Bârzu. Characterization of the calmodulin-binding and of the catalytic domain of bordetella pertussis adenylate cyclase. J Biol Chem, 264:4015-4020, 1989.
[50] D A Case, T A Darden, T E Cheatham, C L Simmerling, J Wang, R E Duke, K M Merz, B Wang, D A Pearlman, M Crowley, S Brozell, V Tsui, H Gohlke, J Mongan, V Hornak, G Cui, P Beroza, J W Schafineister, Cand Caldwell, W S Ross, and P A. Kollman. AMBER 9. 2004.
[51] R J Loncharich, B R Brooks, and R W Pastor. Langevin dynamics of peptides: the frictional dependence of isomerization rates of N-acetylalanyl-N′-methylamide. Biopolymers, 32:523-535, 1992.
[52] Y. Shen, N. L. Zhukovskaya, Q. Guo, J. Florin, and W. J. Tang. Calcium-independent calmodulin binding and two-metal-ion catalytic mechanism of anthrax edema factor. EMBO J., 24:929-941, March 2005.
[53] M. Rarey. FlexX release 2.1 User Guide. Technical report, BioSolveIt, 2007.

We claim:

1. A method for identifying one or more compounds that inhibit the activation of a biomolecule, comprising
(a) identifying an initial inactive conformation of the biomolecule, a final active conformation of the biomolecule and one or more intermediate conformations of the biomolecule between its initial and final conformation;
(b) identifying one or more pockets on the biomolecule, in said one or more intermediate conformations;
(c) screening said one or more pockets with a screening library of compounds; and
(d) selecting one or more compounds that inhibit the activation of the biomolecule

2. The method according to claim 1, wherein the pockets identified at step (b) are other than the functional active site of the biomolecule.

3. A method for identifying one or more compounds that inhibit the activation between a biomolecule, said method comprising:
(a) determining a transition path describing the conformational change of the biomolecule during its activation, said transition path having an initial point and a final point, where the initial point is the inactive conformation of the biomolecule and the final point is the active conformation of the biomolecule;
(b) refining the transition path describing the conformational change of the biomolecule during its activation;
(c) identifying intermediate conformations of the biomolecule in the transition path;
(d) identifying one or more pockets on the biomolecule;
(e) screening said one or more pockets with a screening library of compounds; and
(f) selecting one or more compounds that inactivates the biomolecule.

4. The method of claim 3, wherein the identifying step (c) is performed before the refining step (b).

5. The method of claim 3, wherein the biomolecule is a protein.

6. The method of claim 3, wherein said screening comprises using a docking algorithm.

7. The method of claim 6, wherein the docking algorithm proceeds through incremental construction of said compound in said one or more pockets.

8. The method of claim 7, wherein the docking algorithm generates a scoring function.

9. The method of claim 8, wherein the scoring function is used to select compounds in a first, inactive, conformation.

10. The method of claim 8, wherein the scoring function is used to discard compounds in a second, intermediate, conformation.

11. The method of claim 6, wherein the docking algorithm generates one or more structural interaction fingerprints.

12. The method of claim 11, wherein the one or more structural interaction fingerprints are evaluated using their Tanimoto coefficients.

13. The method of claim 3, wherein said biomolecule is Edema Factor (EF) or Adenylate Cyclase Toxin (CyaA).

14. The method of claim 3, wherein said one or more pockets comprises the SABC pocket of EF.

15. The method of claim 3, wherein said refining comprises a reduction of the number of conformations in the transition path.

16. The method of claim 15, wherein said refining comprises identifying transition sub-paths that do not have an energy above a threshold of from about 200 kcal/mol to about 4000 kcal/mol above the minimum energy of the path.

17. The method of claim 15, wherein said refining comprises identifying transition sub-paths that do not have an energy above a threshold of from about 500 kcal to about 3000 kcal/mol above the minimum energy of the path.

18. The method of claim 3, wherein said refining comprises sampling of intermediate conformations of the biomolecule in the transition path.

19. The method of claim 3, wherein said refining comprises at least one cycle comprising the steps of:
a) selecting a pair of intermediate conformations and
b) identifying one or more by-passes between said selected pair of intermediate conformations, wherein each by-pass comprises a new sequence of intermediate conformations.

20. The method of claim 19, further comprising selecting by-passes.

21. The method of claim 20, further comprising assembling the selected by-passes.

22. The method of claim 19, further comprising reducing the number of intermediate conformations of the biomolecule in the transition.

23. The method of claim 22, wherein the number of intermediate conformations is reduced by retaining intermediate conformations between which there is no energy barrier above a given threshold.

24. A method for reducing the risk of Bacillus anthracis infection comprising administering to a patient at risk of infection a therapeutically effective amount of a compound of the formula (I): 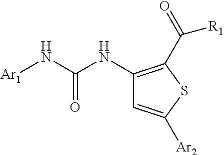
or a pharmaceutically acceptable salt thereof;
wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

25. The method of claim 24, wherein Ar₁ is phenyl.

26. The method of claim 24, wherein Ar₂ is phenyl.

27. The method of claim 24, wherein Ar₂ is substituted with one or more halogen groups or one or more alkoxy groups.

28. The method of claim 27, wherein said one or more halogen groups are bromine, chlorine or fluorine.

29. The method of claim 24, wherein R₁ is hydroxy or alkoxy.

30. The method of claim 24, wherein the compound of the formula (I) is a compound of the formula:

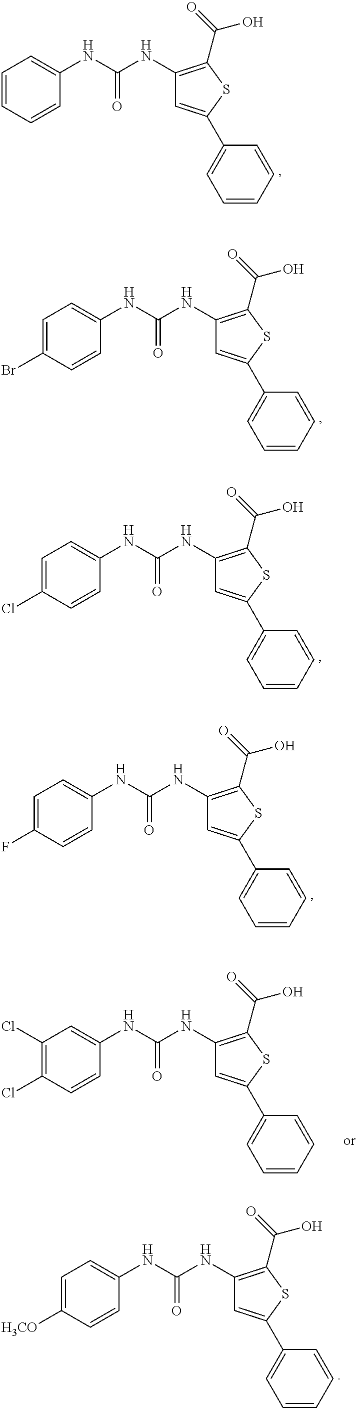

31. The method of claim 24, wherein the compound of the formula (I) is a compound of the formula:

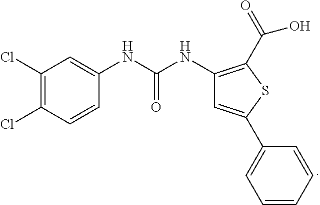

32. A method for reducing the risk of a Bordetella pertussis infection comprising administering to a patient at risk of infection a therapeutically effective amount of a compound of the formula (I): 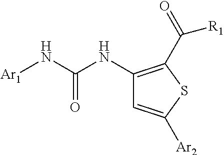
or a pharmaceutically acceptable salt thereof;
wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

33. The method of claim 32, wherein Ar₁ is phenyl.

34. The method of claim 32, wherein Ar₂ is phenyl.

35. The method of claim 32, wherein Ar₂ is substituted with one or more halogen groups or one or more alkoxy groups.

36. The method of claim 32, wherein said one or ore halogen groups are bromine, chlorine or fluorine.

37. The method of claim 32, wherein R₁ is hydroxy or alkoxy.

38. The method of claim 32, wherein the compound of the formula (I) is a compound of the formula:

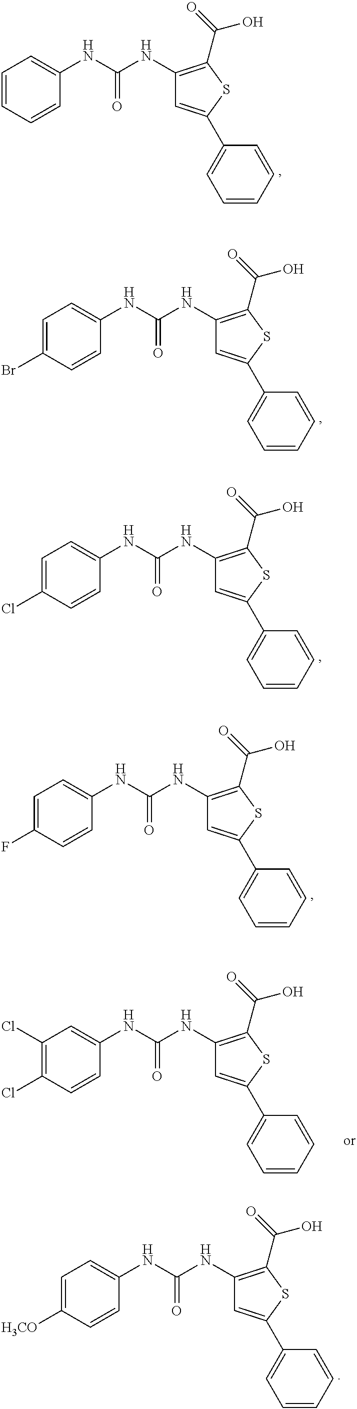

39. The method of claim 32, wherein the compound of the formula (I) is a compound of the formula:

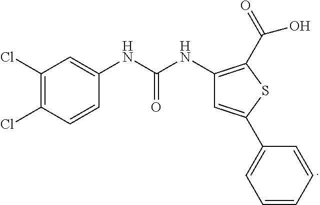

40. A complex between Edema Factor and a compound of the formula (I): 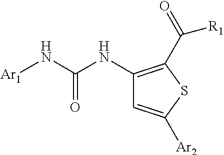
or a pharmaceutically acceptable salt thereof;
wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

41. A compound identified by the method of claim 1.

42. The compound of claim 41, wherein said compound comprises a thiophenyl ureido acid scaffold.

43. A method of inhibiting Edema Factor (EF) comprising contacting EF with a compound of the formula (I): 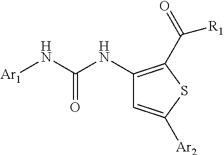
or a pharmaceutically acceptable salt thereof;
wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy.

44. A pharmaceutical composition comprising a compound of the formula (I): 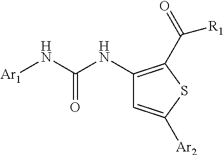
or a pharmaceutically acceptable salt thereof;
wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy; and
a pharmaceutically acceptable carrier or excipient.

45. A kit comprising a pharmaceutical composition comprising a compound of the formula (I): 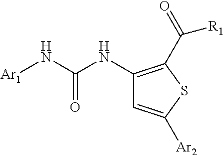
or a pharmaceutically acceptable salt thereof;
wherein:
Ar₁ is an optionally substituted aromatic ring;
Ar₂ is an optionally substituted aromatic ring, where Ar₁ and Ar₂ are the same or different; and
R₁ is hydroxy, alkoxy, arylalkyleneoxy, or heteroarylalkyleneoxy; and
a pharmaceutically acceptable carrier or excipient.
